(19) United States
(12) Patent Application Publication
(10) Pub. No.: US 2022/0177512 A1
(43) Pub. Date: Jun. 9, 2022

(54) CYCLIC PEPTIDE PRODUCTION METHOD

(71) Applicant: AJINOMOTO CO., INC., Tokyo (JP)

(72) Inventors: Daisuke TAKAHASHI, Kawasaki-shi (JP); Tatsuji INOMATA, Yokkaichi-shi (JP)

(73) Assignee: AJINOMOTO CO., INC., Tokyo (JP)

(21) Appl. No.: 17/645,836

(22) Filed: Dec. 23, 2021

Related U.S. Application Data
(63) Continuation of application No. PCT/JP2020/025157, filed on Jun. 26, 2020.

(30) Foreign Application Priority Data
Jun. 28, 2019 (JP) 2019-122174

(51) Int. Cl. C07K 1/06; C07K 7/52

(52) U.S. Cl. CPC C07K 1/063; C07K 7/52

(57) ABSTRACT

A method for producing a cyclic peptide, including the following step (1) and step (2):

(1) a step of cyclizing a linear peptide; and

(2) a step of obtaining a cyclic peptide by adding a poor solvent to a mixture of the cyclic peptide and multimeric impurity, which mixture is obtained in the above-mentioned step, and filtering off the multimeric impurity as an insoluble material can efficiently remove multimeric impurity by-produced during a cyclization reaction, improve the purity of the obtained cyclic peptide, and reduce a burden on the purification step.

### CROSS REFERENCES TO RELATED APPLICATIONS

This application is a continuation of International Patent Application No. PCT/JP2020/025157, filed on Jun. 26, 2020, and claims priority to Japanese Patent Application No. 2019-122174, filed on Jun. 28, 2019, both of which are incorporated herein by reference in their entireties.

### BACKGROUND OF THE INVENTION

### Field of the Invention

The present invention relates to production methods of cyclic peptides.

### Discussion of the Background

In recent years, drug discovery development aiming at improving blood stability and the like by cyclizing peptide has been actively performed, and various forms of cyclic peptides have been reported. As an intramolecular S—S-cyclized S—S type cyclic peptide in which the SH groups in the side chains of cysteine, which is a constituent amino acid, and cysteine derivatives are linked to each other by disulfide bonds, the SH group extending from the N-terminal of the peptide chain is linked to the SH group in the side chain of cysteine or a cysteine derivative by a disulfide bond, somatostatin, octreotide, linaclotide, plecanatide, ziconotide, atosiban, eptifibatide and the like are known. In addition, cyclosporine, cyclic RGD peptide and the like are known as lactam-type cyclic peptides in which an amino group and a carboxyl group at the terminal or in the side chain of the peptide chain are linked by an amide bond. Furthermore, peptide compounds with cyclic thioether bonds such as carbetocin, barusiban, merotocin and the like are known as C—S type cyclic peptides with a carbonylalkylene group between the N-terminal of the peptide chain, and the side chain SH group of cysteine, which is a constituent amino acid, or a cysteine derivative.

However, when conventional production methods are applied to any of the cyclic peptides, multimeric impurities bonded between molecules such as dimer, trimer, oligomer, and the like are by-produced during a cyclization reaction, and the yield of the cyclic peptide of interest becomes low. Moreover, it was difficult to remove these multimeric impurities. Therefore, to suppress the by-production of multimeric impurities, a method having extremely low production efficiency, such as carrying out a cyclization reaction under dilute conditions, and the like is generally adopted. To remove multimeric impurities, WO 2017/134687, which is incorporated herein by reference in its entirety, describes that, in a peptide having two or more SH groups in a molecule, all protecting groups at the N-terminal and C-terminal of the peptide chain and in the side chains of the constituent amino acids are removed, and then intramolecular S—S cyclization is performed in an aqueous solvent under oxidizing conditions, the peptide is treated at a low temperature under acidic conditions (pH 2 to 4) to cause precipitation of dimer, trimer and the like, which are non-polar high-molecular-weight impurities, and the above-mentioned impurities are removed by centrifugation or filtration.

### SUMMARY OF THE INVENTION

Accordingly, it is one object of the present invention to provide a production method of a high-purity cyclic peptide by efficiently removing multimeric impurities by-produced during a cyclization reaction.

This and other objects, which will become apparent during the following detailed description, have been achieved by the inventors' efficiently obtaining cyclic peptide at a high purity by precipitating multimeric impurities produced when cyclizing linear peptide by adding a poor solvent, and filtering them off as insoluble materials.

That is, the present invention has the following characteristics.
(1) A method for producing a cyclic peptide, comprising the following step (1) and step (2):
   (1) a step of cyclizing a linear peptide; and
   (2) a step of obtaining a cyclic peptide by adding a poor solvent to a mixture of the cyclic peptide and multimeric impurity, which mixture is obtained in the above-mentioned step, and filtering off the multimeric impurity as an insoluble material.
(2) The production method of (1), comprising further adding a good solvent before, simultaneously with, or after addition of the above-mentioned poor solvent.
(3) The production method of (1) or (2), wherein the cyclic peptide has a cyclic structure of either a) S—S type, b) lactam type, c) C—S type, d) C—C type or e) lactone type.
(3′) The production method of (1) or (2), wherein the cyclic peptide has a cyclic structure of either a) S—S type, b) lactam type, c) C—S type or d) C—C type.
(4) The production method of any of (1) to (3) and (3′), wherein, in the linear peptide, the C-terminal is protected by a protecting group, and cyclization occurs between i) side chains of constituent amino acids or ii) the N-terminal and any of the side chains of the constituent amino acids.
(5) The production method of (4), wherein the protecting group of the C-terminal and/or protecting groups of the side chains of the constituent amino acids of the linear peptide are liquid phase protecting groups or pseudo solid phase protecting groups.
(6) The production method of (4), wherein, in the above-mentioned step (1), the protecting group of the C-terminal of the linear peptide is a solid phase carrier, and the method further comprises a step of deprotecting the solid phase carrier alone before the above-mentioned step (2).
(7) The production method of any of (4) to (6), wherein the poor solvent is a solvent capable of precipitating/depositing multimeric impurities by-produced when obtaining a cyclic peptide by cyclizing the above-mentioned linear peptide.
(8) The production method of (7), wherein the good solvent is a solvent capable of dissolving the above-mentioned cyclic peptide of interest.
(9) The production method of any of (1) to (3) and (3′), wherein, in the linear peptide, the C-terminal is not protected, parts other than the part to be cyclized are protected, and cyclization occurs between i) side chains of constituent amino acids, ii) the N-terminal and a side chain of a constituent amino acid, iii) the C-terminal and a side chain of a constituent amino acid, or iv) the N-terminal and the C-terminal.
(9A) The production method of (4) or (9), further comprising
   a step of removing all protecting groups of the cyclic peptide obtained in step (1),
      between step (1) and step (2).
(10) The production method of (9), wherein a protecting group of the side chain of the constituent amino acid of the linear peptide is a liquid phase protecting group or a pseudo solid phase protecting group.
(11) The production method of (9) or (10), wherein the poor solvent is a solvent capable of precipitating/depositing multimeric impurities by-produced when obtaining a cyclic peptide by cyclizing the above-mentioned linear peptide.
(12) The production method of (11), wherein the good solvent is a solvent capable of dissolving the above-mentioned cyclic peptide of interest.
(13) The production method of any of (1) to (3) and (3′), wherein, in the linear peptide, all of the C-terminal, the N-terminal, and the side chains of the constituent amino acids are not protected, and cyclization occurs between i) side chains of constituent amino acids, ii) the N-terminal and a side chain of a constituent amino acid, iii) the C-terminal and a side chain of a constituent amino acid, or iv) the N-terminal and the C-terminal.
(14) The production method of (13), further comprising
   a step of isolating the cyclic peptide obtained in step (1),
      between step (1) and step (2).
(15) The production method of (13) or (14), wherein the poor solvent is a solvent capable of precipitating/depositing multimeric impurities by-produced when obtaining a cyclic peptide by cyclizing the above-mentioned linear peptide.
(16) The production method of (15), wherein the good solvent is a solvent capable of dissolving the above-mentioned cyclic peptide of interest.
(17) The production method of any of (1) to (16) and (3′), further comprising
   (3) a step of removing all protecting groups after the above-mentioned step (2).
(18) A method for producing a peptide having a cyclic thioether bond, comprising any of the following steps:
   (A) a step of cyclizing a linear peptide in which the C-terminal of the linear peptide is or is not protected by a protecting group, the N-terminal is modified with an alkylenecarbonyl group having a leaving group, and a side chain of constituent amino acid cysteine or cysteine derivative is not protected, between the N-terminal and the side chain of the cysteine or cysteine derivative,
   (B) a step of cyclizing a linear peptide in which the C-terminal of the linear peptide is or is not protected by a protecting group, the N-terminal is or is not protected, and a side chain of constituent amino acid cysteine or cysteine derivative is modified with an alkylene group having a carboxy group, between the N-terminal and the side chain of the cysteine or cysteine derivative, after deprotection of the N-terminal when it is protected, or
   (C) a step of cyclizing a linear peptide in which the C-terminal of the linear peptide is or is not protected by a protecting group, an amino group of a side chain of a constituent amino acid is or is not protected, and a side chain of constituent amino acid cysteine or cysteine derivative is modified with an alkylene group having a carboxy group, between the side chain of the constituent amino acid having the amino group and the side chain of the cysteine or cysteine derivative, after deprotection of the amino group of the side chain of the constituent amino acid when it is protected.
(18′) A method for producing a peptide having a cyclic thioether bond, comprising any of the following steps:
   (A) a step of cyclizing a linear peptide in which the C-terminal of the linear peptide is protected by a protecting group, the N-terminal is modified with an alkylenecarbonyl group having a leaving group, and a side chain of constituent amino acid cysteine or cysteine derivative is not protected, between the N-terminal and the side chain of the cysteine or cysteine derivative,
   (B) a step of cyclizing a linear peptide in which the C-terminal of the linear peptide is protected by a protecting group, the N-terminal is not protected, and a side chain of constituent amino acid cysteine or cysteine derivative is modified with an alkylene group having a carboxy group, between the N-terminal and the side chain of the cysteine or cysteine derivative, or
   (C) a step of cyclizing a linear peptide in which the C-terminal of the linear peptide is protected by a protecting group, an amino group of a side chain of a constituent amino acid is not protected, and a side chain of constituent amino acid cysteine or cysteine derivative is modified with an alkylene group having a carboxy group, between the side chain of the constituent amino acid having the amino group and the side chain of the cysteine or cysteine derivative.
(19) The production method of (18) or (18′), wherein the protecting group of the C-terminal of the linear peptide is a pseudo solid phase protecting group, a liquid phase protecting group, or a solid phase carrier.
(20) The production method of (18) or (19), wherein the step (B) comprises (B-1) a step of producing a linear peptide in which the C-terminal of the linear peptide is or is not protected by a protecting group, the N-terminal is or is not protected, and a side chain of constituent amino acid cysteine or cysteine derivative is modified with an alkylene group having a carboxy group by modifying, with an alkylene group having a carboxy group, a side chain of the constituent amino acid cysteine or cysteine derivative of the linear peptide in which the C-terminal of the linear peptide is or is not protected by a protecting group, the N-terminal is or is not protected, and the side chain of the cysteine or cysteine derivative is not protected, or
   the step (C) comprises (C-1) a step of producing a linear peptide in which the C-terminal of the linear peptide is or is not protected by a protecting group, an amino group of a side chain of a constituent amino acid is or is not protected, and a side chain of constituent amino acid cysteine or cysteine derivative is modified with an alkylene group having a carboxy group by modifying, with an alkylene group having a carboxy group, a side chain of constituent amino acid cysteine or cysteine derivative of a linear peptide in which the C-terminal of the linear peptide is or is not protected by a protecting group, an amino group of a side chain of a constituent amino acid is or is not protected, and the side chain of the cysteine or cysteine derivative is not protected.
(21) The production method of any of (18) to (20), wherein the step (B) comprises (B-2) a step of removing all protecting groups of the obtained protected cyclic peptide.

### Advantageous Effects of Invention

According to the present invention, a method for producing a cyclic peptide that can efficiently remove multimeric impurity by-produced during a cyclization reaction, improve the purity of the obtained cyclic peptide, and reduce a burden on the purification step can be provided.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows the outline of the embodiments of the present invention. In the Figure, step (1) to step (3) in the “Embodiment 1”, “Embodiment 2”, and “Embodiment 3” correspond to step (1) to step (3) in the present invention. The embodiment of the known example corresponds to the embodiment of patent document 1 in the present DESCRIPTION.

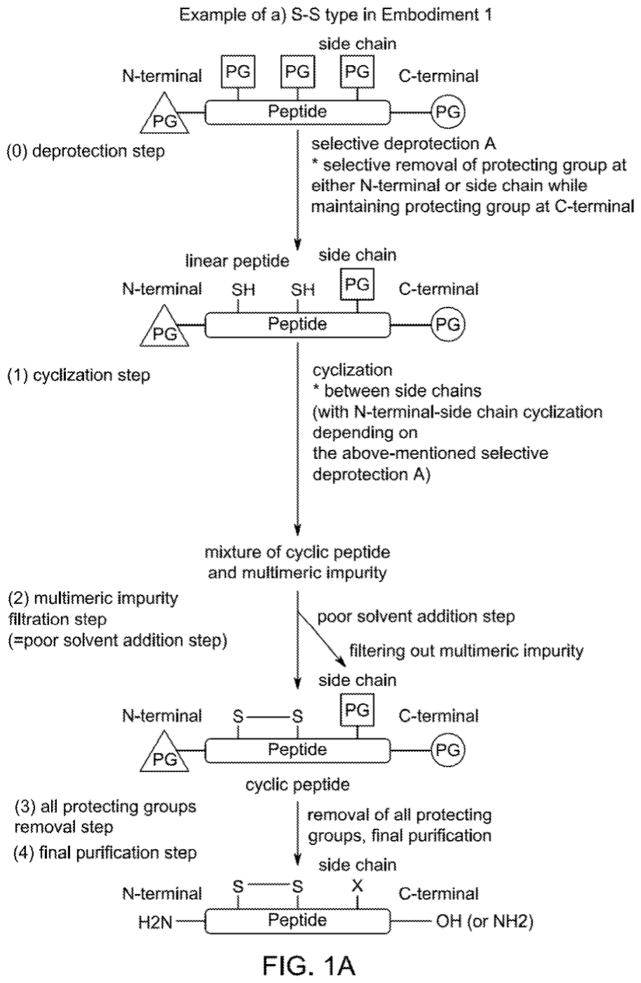

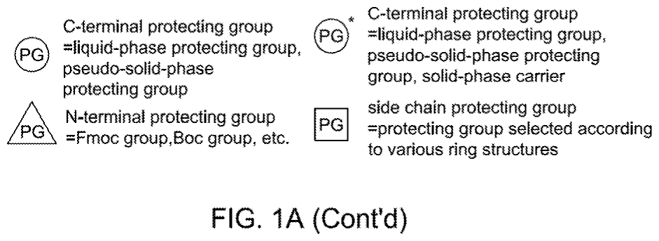

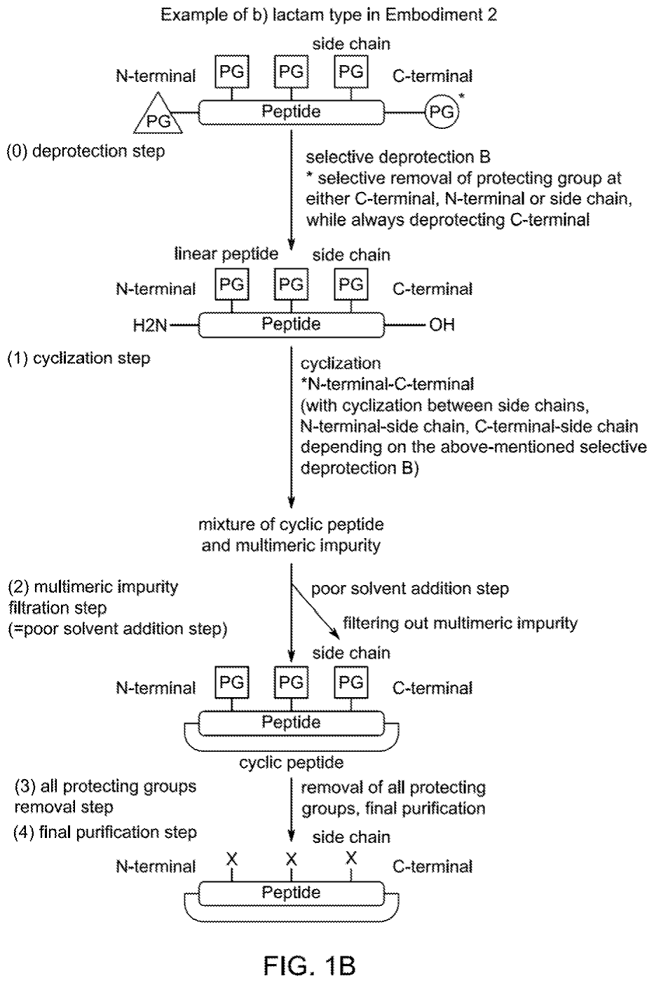

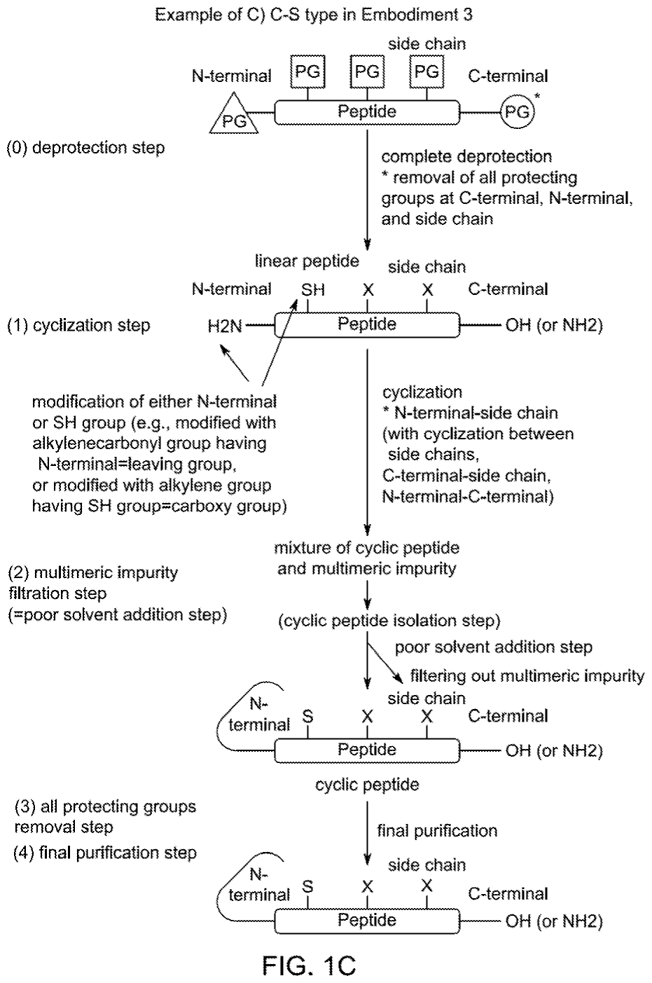

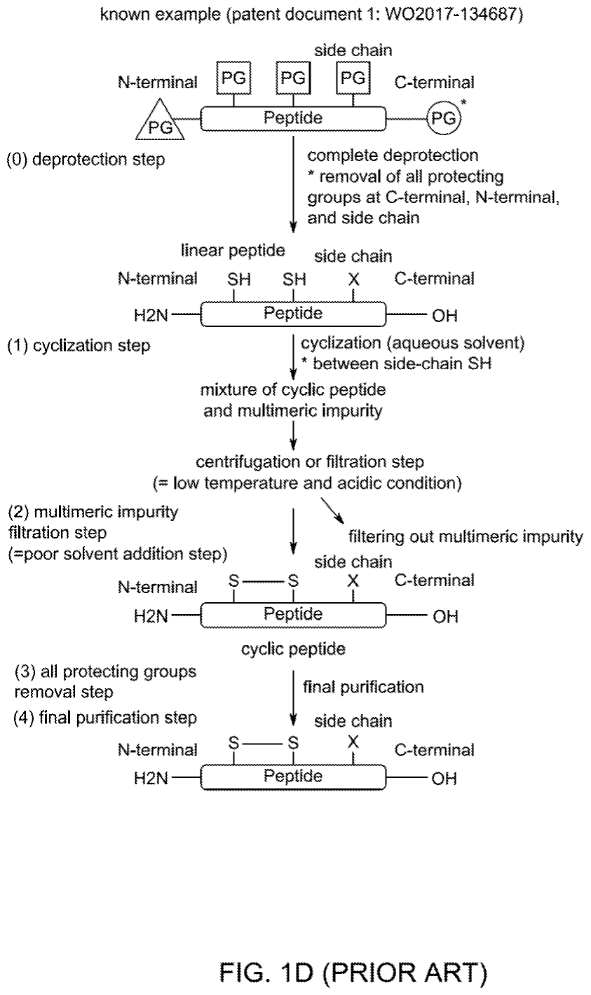

### DETAILED DESCRIPTION OF THE PREFERRED EMBODIMENTS

Unless otherwise specified in the following sentences, any technical terms and scientific terms used in the present specification, have the same meaning as those generally understood by those of ordinary skill in the art the present invention belongs to. Any methods and materials similar or equivalent to those described in the present specification can be used for practicing or testing the present invention, and preferred methods and materials are described in the following. All publications and patents referred to in the present specification are hereby incorporated by reference so as to describe and disclose constructed products and methodology described in, for example, publications usable in relation to the described invention.

The “cyclic peptide” in the present invention is a peptide having a cyclic chemical structure formed by the binding of the constituent amino acids, and may have, as part of the cyclic structure, a partial structure other than that derived from the constituent amino acids. Examples of the partial structure other than that derived from the constituent amino acids include carbonylalkylene, carbonylalkylenethio and the like.

When practicing the present invention, the kind of the cyclic peptide to be the target is not particularly limited, and it may be, for example, a pharmaceutical product. In addition, it may be a naturally occurring substance, or a non-naturally occurring substance. Examples of such cyclic peptide include, but are not limited to, somatostatin, octreotide, linaclotide, plecanatide, ziconotide, atosiban, eptifibatide, and the like as S—S type cyclic peptide; cyclosporin, and the like as lactam type cyclic peptide; and carbetocin, barusiban, merotocin, and the like as C—S type cyclic peptide.

An amino acid to be a constituent unit of a peptide produced by the method of the present invention is a compound having an amino group and a carboxy group in the same molecule, and may be a natural amino acid or non-natural amino acid, and an L form, a D form or a racemate. A peptide is synthesized by repeating a dehydration condensation step (condensation step) of an amino group of an amino acid component and a carboxy group of other amino acid component, according to the amino acid sequence of the peptide.

The production method of the present invention is explained below. The method for producing a cyclic peptide of the present invention characteristically includes the following step (1) and step (2):
(1) a step of cyclizing a linear peptide; and
(2) a step of obtaining a cyclic peptide by adding a poor solvent to a mixture of the cyclic peptide and multimeric impurity which mixture is obtained in the above-mentioned step, and filtering off the multimeric impurity as an insoluble material.
   Step (1): a step of cyclizing a linear peptide

The “linear peptide” in the present invention is not particularly limited as long as it optionally has a protecting group and/or a pseudo solid phase protecting group in the N-terminal, C-terminal and/or the side chains of constituent amino acids, and has the unprotected N-terminal, C-terminal and/or side chains of constituent amino acids, that can cause a cyclization reaction. The linear peptide also includes one showing partial cyclization.

The cyclization occurs when unprotected N-terminal, C-terminal and/or side chains of constituent amino acids capable of causing a cyclization reaction are bonded to each other to form, for example, —S—S— bond, —CO—NH— bond, —C—S— bond, —C—C— bond, —CO—O— bond and the like.

Examples of the —S—S— bond include a bond between “thiol groups on the side chains of constituent amino acids”, a bond between “the N-terminal modified with an alkylenecarbonyl group having a thiol group” and “a thiol group on the side chain of constituent amino acid”, a bond between “an amino group on the side chain of constituent amino acid modified with an alkylenecarbonyl group having a thiol group” and “a thiol group on the side chain of constituent amino acid”, and the like.

As used herein, the “alkylenecarbonyl group having a thiol group” is, for example, a group that reacts with the thiol group on the side chain of the constituent amino acid cysteine or cysteine derivative. Examples of the alkylene in the above-mentioned alkylenecarbonyl group having a thiol group include an alkylene group having 1 to 6 carbon atoms, more preferably an alkylene group having 1 to 3 carbon atoms, and a methylene group, an ethylene group and a propylene group can be mentioned.

Examples of the —CO—NH— bond include a bond between the N-terminal and the C-terminal, a bond between the N-terminal and “a carboxyl group on the side chain of constituent amino acid”, a bond between “an amino group on the side chain of constituent amino acid” and the C-terminal, a bond between “an amino group on the side chain of constituent amino acid” and “a carboxyl group on the side chain of constituent amino acid”, and the like.

Examples of the —C—S— bond include a bond between “a thiol group on the side chain of constituent amino acid” and “the N-terminal modified with an alkylenecarbonyl group having a leaving group”, a bond between “a thiol group on the side chain of constituent amino acid” and “an amino group on the side chain of constituent amino acid modified with an alkylenecarbonyl group having a leaving group”, and the like.

As used herein, the “alkylenecarbonyl group having a leaving group” is, for example, a group that reacts with a thiol group on the side chain of constituent amino acid cysteine or cysteine derivative, and a halogenoalkylenecarbonyl group, a tosyloxyalkylenecarbonyl group, a mesyloxyalkylenecarbonyl group and the like can be mentioned. As the halogenoalkylenecarbonyl group, a chloroalkylenecarbonyl group, a bromoalkylenecarbonyl group, an iodoalkylenecarbonyl group and the like can be mentioned. Among these, a chloroalkylenecarbonyl group is more preferred. As the alkylene group of the above-mentioned halogenoalkylenecarbonyl group, tosyloxyalkylenecarbonyl group, and mesyloxyalkylenecarbonyl group, an alkylene group having 1 to 6 carbon atoms can be mentioned, an alkylene group having 1 to 3 carbon atoms is more preferred, and a methylene group, an ethylene group, and a propylene group can be mentioned.

Examples of the —C—C— bond include a bond between “the side chains of constituent amino acids modified with a terminal olefin group”, and the like.

Examples of the —CO—O— bond include a bond between the C-terminal and “a hydroxy group on the side chain of constituent amino acid”, a bond between “a hydroxy group on the side chain of constituent amino acid” and “a carboxyl group on the side chain of constituent amino acid”, and the like.

The cyclization reaction can be carried out under the conditions generally used in the art.

In the case of an —S—S— bond, for example, conditions generally used for an oxidation reaction between thiol groups, and the like can be mentioned.

In the case of a —CO—NH— bond, for example, conditions generally used when forming an intramolecular amide bond (lactam bond), and the like can be mentioned.

In the case of a —C—S— bond, for example, conditions generally used when reacting an alkyl group having a leaving group with a thiol group, and the like can be mentioned.

In the case of a —C—C— bond, for example, conditions generally used when utilizing an olefin metathesis reaction (e.g., Org. Lett., 2015, 17 (3), 696) and the like can be mentioned.

In the case of a —CO—O— bond, for example, conditions generally used when forming an intramolecular ester bond (lactone bond), and the like can be mentioned.

From the above, a cyclic peptide can be produced in which the cyclic structure of the cyclic peptide is either a) S—S type, b) lactam type, c) C—S type, d) C—C type, or e) lactone type.

As the reaction solvent in the cyclization reaction, a solvent capable of dissolving cyclic peptide is preferred.

In the “totally protected peptide” in which the N-terminal, C-terminal and/or side chains of the constituent amino acids are all protected in the linear peptide of step (1), it is necessary to deprotect the site to be cyclized in this step (1). For deprotecting the site to be cyclized in this step (1), a deprotection method known per se can be adopted without particular limitation, according to the kind of the protecting group to be removed. When deprotecting the site to be cyclized in this step (1), it is necessary to deprotect only the site to be cyclized in this step (1) among the “totally protected peptide”, and deprotection conditions with such selectivity can be appropriately selected in deprotection. Those skilled in the art can appropriately select appropriate conditions based on the overall synthetic strategy. As the conditions for each deprotection, the conditions described in the following step (3) deprotection step can be used.

By cyclizing the linear peptide, a mixture of cyclic peptide and multimeric impurities as by-products thereof can be obtained.

As used herein, the “cyclic peptide” is an intramolecularly cyclized peptide that is the substance of interest.

The “multimeric impurity” is a by-product when a cyclic peptide is obtained by cyclizing a linear peptide, and includes intermolecularly cyclized peptide multimers (dimer, trimer, oligomer, polymer etc.), and intermolecularly bonded linear peptide multimers (dimer, trimer, oligomer, polymer etc.) as precursor substances thereof.

Step (2): step of obtaining a cyclic peptide by adding a poor solvent to a mixture of the cyclic peptide and multimeric impurity, and filtering off the multimeric impurity as an insoluble material

The “poor solvent” is a solvent capable of precipitating/depositing multimeric impurities by-produced when obtaining cyclic peptide by cyclizing the linear peptide, and is not particularly limited. For example, acetonitrile, IPE (diisopropyl ether), diethyl ether, toluene, hexane, heptane, methanol, ethanol, isopropyl alcohol, THF (tetrahydrofuran), water and the like can be mentioned. Only one kind of these may be used, or a mixture of two or more kinds thereof may be used.

A good solvent may be further added before, simultaneously with, or after addition of the above-mentioned poor solvent.

The “good solvent” is a solvent capable of dissolving the cyclic peptide of interest, and is not particularly limited. For example, chloroform, dichloromethane, DMF (dimethylformamide), N-methylpyrrolidone, methanol, ethanol, isopropyl alcohol, THF (tetrahydrofuran) and the like can be mentioned. Only one kind of these may be used, or a mixture of two or more kinds thereof may be used. As the good solvent, the reaction solvent used in the cyclization step of step (1) is preferred. A good solvent is preferably added before or simultaneously with the addition of the poor solvent in step (2). It is desirable that the cyclic peptide of interest is completely dissolved when the poor solvent is added; however, the form of a slurry is also included.

The combination of these good solvent and poor solvent may be selected such that there is a greater difference in solubility between the cyclic peptide of interest and the multimeric impurity as a by-product.

It is preferable that the above-mentioned “poor solvent” and the above-mentioned “good solvent” are not the same solvent.

### Protecting Group of C-Terminal of Peptide

In the present specification, the protecting group of the C-terminal of peptide includes, for example, liquid phase protecting groups and pseudo solid phase protecting groups. It is not particularly limited, and the protecting groups generally used in the art can be mentioned. For example, an ester-type protecting group, an amide-type protecting group, a hydrazide-type protecting group, and the like can be mentioned.

As the ester-type protecting group, substituted or unsubstituted alkyl ester, and substituted or unsubstituted aralkyl ester are preferably used. As the substituted or unsubstituted alkyl ester, methyl ester, ethyl ester, tert-butyl ester, cyclohexyl ester, trichloroethyl ester, phenacyl ester and the like are preferably used. As the substituted or unsubstituted aralkyl ester, benzyl ester, p-nitrobenzyl ester, p-methoxybenzyl ester, diphenylmethyl ester, 9-fluorenylmethyl (Fm) ester, 4-picolyl (Pic) ester and the like are preferably used.

As the amide-type protecting group, unsubstituted amide, primary amide such as N-methylamide, N-ethylamide, N-benzylamide and the like, secondary amide such as N,N-dimethylamide, pyrrolidinylamide, piperidinylamide and the like, and the like are preferably used.

As the hydrazide-type protecting group, unsubstituted hydrazide, N-phenylhydrazide, N,N′-diisopropylhydrazide and the like are preferably used.

### Protecting Group of N-Terminal of Peptide

In the present specification, the protecting group of the N-terminal of peptide is not particularly limited and includes, for example, protecting groups generally used in the art. For example, a 9-fluorenylmethyloxycarbonyl group (Fmoc group), a benzyloxycarbonyl group (Cbz group), a tert-butoxycarbonyl group (Boc group) and the like can be mentioned. It is preferably an Fmoc group.

### Protecting Group for Functional Group on Peptide

In the present specification, the protecting group for the side chain of the peptide is not particularly limited, and examples thereof include the protecting groups described in PEPTIDE GOUSEI NO KISO TO JIKKEN, published by Maruzen Co., Ltd. (1985), PROTECTIVE GROUPS IN ORGANIC SYNTHESIS, the third edition, published by JOHN WILLY&SONS (1999) and the like, which are incorporated herein by reference it their entireties.

When the side chain is a carboxy group, the same protecting group as described above can be mentioned as the protecting group of the C-terminal. Examples thereof include a liquid phase protecting group, a pseudo-solid phase protecting group, and a solid phase carrier.

When the side chain is an amino group, a urethane-type protecting group, an acyl-type protecting group, a sulfonyl-type protecting group and the like can be mentioned.

As the urethane-type protecting group, for example, a methoxycarbonyl group, an ethoxycarbonyl group, a tert-butoxycarbonyl (Boc) group, a benzyloxycarbonyl (Z) group, and the like are used, and a methoxycarbonyl group, an ethoxycarbonyl group, a Boc group, and the like are preferred.

As the acyl-type protecting group, for example, a formyl group, an acetyl group, a trifluoroacetyl group and the like are preferably used.

As the sulfonyl-type protecting group, for example, a p-toluenesulfonyl (Ts) group, a p-tolylmethanesulfonyl group, a 4-methoxy-2,3,6-trimethylbenzenesulfonyl group and the like are preferably used.

When the functional group on peptide is a hydroxy group (including phenolic hydroxy group), an alkyl-type protecting group, an alkoxyalkyl-type protecting group, an acyl-type protecting group, an alkylsilyl-type protecting group and the like can be mentioned.

Examples of the alkyl-type protecting group include a methyl group, an ethyl group, a tert-butyl group and the like.

Examples of the alkoxyalkyl-type protecting group include a methoxymethyl group (MOM group), a 2-tetrahydropyranyl group (THP group), an ethoxyethyl group (EE group), and the like.

Examples of the acyl-type protecting group include an acetyl group, a pivaloyl group, a benzoyl group, and the like.

Examples of the alkylsilyl-type protecting group include a trimethylsilyl group (TMS group), a triethylsilyl group (TES group), a tert-butyldimethylsilyl group (TBS group or TBDMS group), a triisopropylsilyl group (TIPS group), a tert-butyldiphenylsilyl group (TBDPS group), and the like.

Other functional groups can also be protected by protecting groups conventionally used in the pertinent technical field. For example, the guanidino group of arginine can be protected by a p-toluenesulfonyl group. The imidazole group of histidine can be protected by a trityl group, a benzyloxymethyl group, and the like. In addition, the indole group of tryptophan can be protected by a formyl group.

While the protecting group for the functional group on peptide is described above, those of ordinary skill in the art can perform this step by appropriately selecting the protecting group according to the protection scheme (e.g., Fmoc/tBu strategy, Boc/Bzl strategy, Bzl/tBu strategy, etc.) selected in the technical field according to the overall synthetic strategy for carrying out the present invention. Among these, the Fmoc/tBu strategy is preferred.

### Liquid Phase Protecting Group

When the present invention is performed under liquid phase conditions, the C-terminal is desirably protected, and when the functional group on peptide is a carboxy group, at least one of the carboxy groups is desirably protected. As the protecting group of the carboxy group, the protecting groups (ester-type protecting group, amide-type protecting group, hydrazide-type protecting group, etc.) recited as the aforementioned “protecting group of C-terminal” can be mentioned. Among these, ester-type protecting group is preferred. As the ester-type protecting group, substituted or unsubstituted alkyl ester, and substituted or unsubstituted aralkyl ester are preferably used. As the substituted or unsubstituted alkyl ester, methyl ester, ethyl ester, tert-butyl ester, cyclohexyl ester, trichloroethyl ester, phenacyl ester and the like are preferably used. As the substituted or unsubstituted aralkyl ester, benzyl ester, p-nitrobenzyl ester, p-methoxybenzyl ester, diphenylmethyl ester, 9-fluorenylmethyl (Fm) ester, 4-picolyl (Pic) ester and the like are preferably used. Particularly, tert-butyl ester, benzyl ester and the like are preferred.

### Pseudo-Solid-Phase Protecting Group

When the present invention is performed under liquid phase conditions, the C-terminal and, when the functional group on peptide is a carboxy group, at least one of the carboxy groups may be protected where necessary by a pseudo-solid-phase protecting group (hereinafter sometimes to be referred to as “anchor” in the present specification) to facilitate purification. While the purification method of a peptide using a pseudo-solid-phase protecting group is not particularly limited, a method known per se (see JP-A-2000-44493, WO 2006/104166, WO 2007/034812, WO 2007/122847, WO 2010/113939, WO 2010/104169, WO 2011/078295, WO 2012/029794, WO 2016/140232, WO 2003/018188, WO 2017/038650, WO 2019/009317, which are incorporated herein by reference in their entireties, and the like) or a method according thereto can be performed. The pseudo-solid-phase protecting group here refers to a group containing an anchor soluble in halogenated solvents or ether solvents, insoluble in polar solvents and having a molecular weight of not less than 300 (e.g., benzyl compound, diphenylmethane compound or fluorene compound), and capable of condensing with a carboxy group.

In the present specification, the pseudo solid phase protecting group is not particularly limited, and a pseudo solid phase protecting group generally used in the art can be mentioned.

Solid Phase Carrier

The “solid phase carrier” may be any solid phase carrier known in the pertinent technical field and suitable for use in solid phase synthesis. In the present specification, the term “solid phase” includes that a peptide is bonded or linked to the above-mentioned solid phase carrier via a conventionally-used functional linker or handle group. A “solid phase” in this context also includes such linker. Examples of the solid phase include polystyrene supports (which may be further functionalized by, for example, p-methylbenzyl-hydrylamine), rigid functionalized supports such as diatomaceous earth-encapsulated polydimethylacrylamide (pepsin K), silica, microporous glass, and the like. The solid phase resin matrix may be composed of an amphiphilic polystyrene-PEG resin or PEG-polyamide or PEG-polyester resin. The solid phase carrier also includes, for example, Wang-PEG resin and Rink-amide PEG resin.

### Isolation Step

A step of isolating the cyclic peptide obtained in step (1) may be further included between the above-mentioned step (1) and step (2). The cyclic peptide obtained in step (1) can be isolated by a method generally used in the art and, for example, filtration and the like can be mentioned. For example, the precipitate may be filtered by adding a solvent that can be used as a poor solvent. As the solvent for filtration, acetonitrile, IPE (diisopropyl ether), diethyl ether, toluene, hexane, heptane, methanol, ethanol, isopropyl alcohol, THF (tetrahydrofuran), water and the like can be mentioned. Only one kind of these may be used, or a mixture of two or more kinds thereof may be used. Preferred are IPE (diisopropyl ether) and diethyl ether. Among others, it is preferable to further include, between the above-mentioned step (1) and step (2), a step of isolating the cyclic peptide obtained in step (1), when, in the linear peptide, all of the C-terminal, N-terminal and side chains of the constituent amino acids are not protected, and cyclization occurs between i) the side chains of the constituent amino acids, ii) N-terminal and the side chain of the constituent amino acid, iii) C-terminal and the side chain of the constituent amino acid or iv) N-terminal and C-terminal.

A step of removing all protecting groups of the cyclic peptide obtained in step (1) may be further included between step (1) and step (2) when, in the linear peptide, the C-terminal is protected by a protecting group, and cyclization occurs between i) side chains of constituent amino acids or ii) the N-terminal and a side chain of a constituent amino acid, or when, in the linear peptide, the C-terminal is not protected, parts other than the part to be cyclized are protected, and cyclization occurs between i) side chains of constituent amino acids, ii) the N-terminal and a side chain of a constituent amino acid, iii) the C-terminal and a side chain of a constituent amino acid, or iv) the N-terminal and the C-terminal.

### Step of Deprotecting All Protecting Groups

### Step (3): Step of Deprotecting All Protecting Groups

A step of removing all protecting groups may be further included after the above-mentioned step (2) in the cases other than when all of the C-terminal, the N-terminal and side chains of the constituent amino acids of the linear peptide used in step (1) are not protected.

For example, in the case of a lower alkyl group such as Me, Et or the like, it can be removed by reaction with a base such as sodium hydroxide, potassium hydroxide or the like in a solvent such as an aqueous organic solvent, a polar organic solvent or the like.

In the case of tBu, it can be removed by reaction with an acid such as trifluoroacetic acid (TFA), hydrochloric acid or the like in a solvent such as chloroform, ethyl acetate or the like.

In the case of Bzl, it can be removed by reaction in a solvent such as methanol, DMF or the like or with a strong acid such as hydrogen fluoride, trifluoromethanesulfonic acid, HBr or the like.

While the acid usable for the removal of a Boc group is not particularly limited, mineral acids such as hydrogen chloride, sulfuric acid, nitric acid and the like, carboxylic acids such as formic acid, trifluoroacetic acid (TFA) and the like, sulfonic acids such as methanesulfonic acid, p-toluenesulfonic acid and the like, or a mixture thereof can be used. As the mixture, for example, hydrogen bromide/acetic acid, hydrogen chloride/dioxane, hydrogen chloride/acetic acid and the like can be mentioned.

While the organic base usable for the removal of an Fmoc group is not particularly limited, secondary amines such as diethylamine, piperidine, morpholine and the like, tertiary amines such as diisopropylethylamine, dimethylaminopyridine, 1,8-diazabicyclo[5.4.0]-7-undecene (DBU), 1,4-diazabicyclo[2.2.2]octane (DABCO), 1,5-diazabicyclo[4.3.0]-5-nonene (DBN) and the like can be mentioned.

More preferably, the Fmoc group is removed by treating same with a non-nucleophilic organic base in a halogenated solvent or ether solvent. The deprotection is performed in a solvent that does not influence the reaction.

Examples of the non-nucleophilic base include 1,8-diazabicyclo[5.4.0]-7-undecene (DBU), 1,4-diazabicyclo[2.2.2]octane (DABCO), 1,5-diazabicyclo[4.3.0]-5-nonene (DBN) and the like. DBU and DBN are preferred, and DBU is more preferred.

Removal of pseudo-solid-phase protecting group is preferably performed by an acid treatment. As an acid to be used for the deprotection, trifluoroacetic acid (TFA), hydrochloric acid, sulfuric acid, methanesulfonic acid, p-toluenesulfonic acid and the like can be mentioned, with preference given to TFA. As a solvent to be used for the deprotection, for example, chloroform, dichloromethane, 1,2-dichloroethane or a mixed solvent thereof and the like can be mentioned. The concentration of an acid to be used for the deprotection is, for example, 0.1 w/v %-5 w/v %.

Removal of the pseudo-solid-phase protecting group may also be performed simultaneously with the removal of the protecting groups of other functional groups in the peptide. In this case, a conventional method used in the field, particularly peptide synthesis, is used, and a method including adding an acid and the like is preferably used. As the acid, trifluoroacetic acid (TFA), hydrochloric acid, sulfuric acid, mesylic acid, tosylic acid, trifluoroethanol, hexafluoroisopropanol and the like are used. Of these, TFA is particularly preferred. The amount of the acid to be used is appropriately set according to the kind of the acid to be used, and an amount suitable for removing the anchor group is used. The amount of the acid to be used is preferably not less than 3 mol, more preferably not less than 5 mol, preferably not more than 100 mol, more preferably not more than 50 mol, per 1 mol of the peptide. Along with such use, trifluoromethanesulfonic acid, trimethylsilyl trifluoromethanesulfonate, BF₃.etherate and the like can also be added as a further source of strong acid.

The conditions for the removal of the above pseudo-solid-phase protecting groups can be appropriately selected by those of ordinary skill in the art according to the kind of the protecting group to be used.

Other protecting groups can be appropriately removed according to the kind thereof by a method generally performed in the pertinent technical field, or according to the deprotection method of the protecting groups described in the present specification.

The above-mentioned steps (1) to (3) can be performed under liquid phase conditions. At that time, those skilled in the art can appropriately select the conditions of the liquid phase according to the synthesis strategy such as the structure of the cyclized peptide of interest and the production purpose (production scale, etc.).

The above-mentioned steps (1) to (3) can also be performed under pseudo solid phase conditions using a pseudo solid phase protecting group. To be specific, it applies when, in the linear peptide, the C-terminal is protected by a protecting group, and cyclization occurs between i) side chains of constituent amino acids or ii) the N-terminal and any of the side chains of the constituent amino acids, and when the protecting groups of the C-terminal and/or the side chains of the constituent amino acids of the linear peptide are pseudo solid phase protecting groups. In addition, it applies when, in the linear peptide, the C-terminal is not protected, parts other than the part to be cyclized are protected, and cyclization occurs between i) side chains of constituent amino acids, ii) the N-terminal and a side chain of the constituent amino acid, iii) the C-terminal and the side chain of the constituent amino acid or iv) the N-terminal and the C-terminal, and when the protecting groups of the side chains of the constituent amino acids of the linear peptide are solid phase protecting groups.

In the above-mentioned case, those skilled in the art can appropriately select the pseudo solid phase conditions under which the pseudo solid phase protecting group is used, according to the synthesis strategy such as the structure of the cyclized peptide of interest and the production purpose (production scale, etc.).

Step (1) can also be performed under solid phase conditions. To be specific, it applies when, in the linear peptide, the C-terminal is protected by a protecting group, and cyclization occurs between i) side chains of constituent amino acids or ii) the N-terminal and a side chain of a constituent amino acid, and when the protecting group of the C-terminal of the linear peptide is a solid phase carrier. In this case, a step of deprotecting the solid phase carrier alone is further contained before the above-mentioned step (2). At that time, those skilled in the art can appropriately select the conditions of solid phase (including deprotection conditions of solid phase carrier), according to the synthesis strategy such as the structure of the cyclized peptide of interest and the production purpose (production scale, etc.).

### Final Purification Step

### Step (4): Final Purification Step

When the cyclic peptide obtained in the above-mentioned steps (1) to (3) is obtained under liquid phase conditions, it can be purified by a method conventionally used in the pertinent technical field.

More specifically, the present invention provides the following Embodiment 1 to Embodiment 3.

### Embodiment 1

A method for producing a cyclic peptide, including the following step (1) and step (2):
(1) a step of cyclizing a linear peptide; and
(2) a step of obtaining a cyclic peptide by adding a poor solvent to a mixture of the cyclic peptide and multimeric impurity, which mixture is obtained in the above-mentioned step, and filtering off the multimeric impurity as an insoluble material,

wherein, in the linear peptide, the C-terminal is protected by a protecting group, and cyclization occurs between i) side chains of constituent amino acids or ii) the N-terminal and a side chain of a constituent amino acid.

In the above, the protecting group of the C-terminal and/or the side chain of the constituent amino acid of the linear peptide is preferably either a liquid phase protecting group or a pseudo solid phase protecting group.

In the above, moreover, the poor solvent is a solvent capable of precipitating/depositing multimeric impurities by-produced when obtaining cyclic peptide by cyclizing the above-mentioned linear peptide, and is preferably at least one selected from acetonitrile, methanol and water.

In the above, moreover, a good solvent may be further added before, simultaneously with, or after addition of the above-mentioned poor solvent. The good solvent is a solvent capable of dissolving the above-mentioned cyclic peptide of interest, and is preferably at least one selected from chloroform, dichloromethane, DMF (dimethylformamide) and THF (tetrahydrofuran).

In another embodiment of Embodiment 1, for example, in step (1), the protecting group of the C-terminal of the linear peptide is a solid phase carrier, and a step of deprotecting the solid phase carrier alone is further included before the above-mentioned step (2).

### Embodiment 2

A method for producing a cyclic peptide, including the following step (1) and step (2):
(1) a step of cyclizing a linear peptide; and
(2) a step of obtaining a cyclic peptide by adding a poor solvent to a mixture of the cyclic peptide and multimeric impurity, which mixture is obtained in the above-mentioned step, and filtering off the multimeric impurity as an insoluble material,

wherein, in the linear peptide, the C-terminal is not protected, parts other than the part to be cyclized are protected, and cyclization occurs between i) side chains of constituent amino acids, ii) the N-terminal and a side chain of a constituent amino acid, iii) the C-terminal and a side chain of a constituent amino acid or iv) the N-terminal and the C-terminal.

In the above, the protecting groups of the side chains of the constituent amino acids of the linear peptide are preferably either a liquid phase protecting group or a pseudo solid phase protecting group.

In the above, moreover, the poor solvent is a solvent capable of precipitating/depositing multimeric impurities by-produced when obtaining cyclic peptide by cyclizing the above-mentioned linear peptide, and is preferably at least one selected from IPE (diisopropyl ether), diethyl ether, toluene, hexane and heptane.

In the above, moreover, a good solvent may be further added before, simultaneously with, or after addition of the above-mentioned poor solvent. The good solvent is a solvent capable of dissolving the above-mentioned cyclic peptide of interest, and is preferably at least one selected from chloroform, dichloromethane, N-methylpyrrolidone and DMF (dimethylformamide).

### Embodiment 3

A method for producing a cyclic peptide, including the following step (1) and step (2):
(1) a step of cyclizing a linear peptide; and
(2) a step of obtaining a cyclic peptide by adding a poor solvent to a mixture of the cyclic peptide and multimeric impurity, which mixture is obtained in the above-mentioned step, and filtering off the multimeric impurity as an insoluble material,

wherein, in the linear peptide, all of the C-terminal, the N-terminal and the side chains of the constituent amino acids are not protected, and cyclization occurs between i) side chains of constituent amino acids, ii) the N-terminal and a side chain of a constituent amino acid, iii) the C-terminal and a side chain of a constituent amino acid or iv) the N-terminal and the C-terminal.

In the above, a step of isolating the cyclic peptide obtained in step (1) is preferably contained between the above-mentioned step (1) and step (2).

In the above, the poor solvent is a solvent capable of precipitating/depositing multimeric impurities by-produced when obtaining cyclic peptide by cyclizing the above-mentioned linear peptide, and is preferably at least one selected from water, IPE (diisopropyl ether), acetonitrile, ethanol, isopropyl alcohol and THF (tetrahydrofuran).

In the above, moreover, a good solvent may be further added before, simultaneously with, or after addition of the above-mentioned poor solvent. The good solvent is a solvent capable of dissolving the above-mentioned cyclic peptide of interest, and is preferably at least one selected from DMF (dimethylformamide), methanol, and N-methylpyrrolidone.

Furthermore, the production method of the present invention is explained.

A method for producing a peptide having a cyclic thioether bond (C—S type cyclic peptide) of the present invention characteristically includes any of the following steps:

(A) a step of cyclizing a linear peptide in which the C-terminal of the linear peptide is or is not protected by a protecting group, the N-terminal is modified with an alkylenecarbonyl group having a leaving group, and a side chain of constituent amino acid cysteine or cysteine derivative is not protected, between the N-terminal and the side chain of the cysteine or cysteine derivative (embodiment A),

(B) a step of cyclizing a linear peptide in which the C-terminal of the linear peptide is or is not protected by a protecting group, the N-terminal is or is not protected, and a side chain of constituent amino acid cysteine or cysteine derivative is modified with an alkylene group having a carboxy group, between the N-terminal and the side chain of the cysteine or cysteine derivative, after deprotection of the N-terminal when it is protected (embodiment B), or

(C) a step of cyclizing a linear peptide in which the C-terminal of the linear peptide is or is not protected by a protecting group, an amino group of a side chain of a constituent amino acid is or is not protected, and a side chain of constituent amino acid cysteine or cysteine derivative is modified with an alkylene group having a carboxy group, between the side chain of the constituent amino acid having the amino group and the side chain of the cysteine or cysteine derivative, after deprotection of the amino group of the side chain of the constituent amino acid when it is protected (embodiment C).

In the present specification, the “cysteine derivative” is, for example, a homocysteine or the like.

In the method for producing a peptide having a cyclic thioether bond of the present invention, the protecting group at the C-terminal of the linear peptide is a pseudo solid phase protecting group, a liquid phase protecting group, or a solid phase carrier. Among these, a pseudo solid phase protecting group is preferred.

The “alkylenecarbonyl group having a leaving group” in the present invention is, for example, a group that reacts with a thiol group on the side chain of constituent amino acid cysteine or cysteine derivative, and a halogenoalkylenecarbonyl group, a tosyloxyalkylenecarbonyl group, a mesyloxyalkylenecarbonyl group and the like can be mentioned. As the halogenoalkylenecarbonyl group, a chloroalkylenecarbonyl group, a bromoalkylenecarbonyl group, an iodoalkylenecarbonyl group and the like can be mentioned. Among these, a chloroalkylenecarbonyl group is more preferred. As the alkylene group of the above-mentioned halogenoalkylenecarbonyl group, tosyloxyalkylenecarbonyl group, and mesyloxyalkylenecarbonyl group, an alkylene group having 1-6 carbon atoms can be mentioned, an alkylene group having 1-3 carbon atoms is more preferred, and a methylene group, an ethylene group, and a propylene group can be mentioned.

The “alkylene group having a carboxy group” in the present invention is, for example, a group that reacts with the N-terminal of the peptide or a group that reacts with the amino group on the side chain of constituent amino acid. As the alkylene group of the above-mentioned “alkylene group having a carboxy group”, an alkylene group having 1 to 6 carbon atoms can be mentioned, an alkylene group having 1 to 3 carbon atoms is more preferred, and a methylene group, an ethylene group, and a propylene group can be mentioned.

Step (B) may further include (B-1) a step of producing a linear peptide in which the C-terminal of the linear peptide is or is not protected by a protecting group, the N-terminal is or is not protected, and a side chain of constituent amino acid cysteine or cysteine derivative is modified with an alkylene group having a carboxy group by modifying, with an alkylene group having a carboxy group, a side chain of the constituent amino acid cysteine or cysteine derivative of the linear peptide in which the C-terminal of the linear peptide is or is not protected by a protecting group, the N-terminal is or is not protected, and the side chain of the cysteine or cysteine derivative is not protected.

Step (B) may further include (B-2) a step of removing all protecting groups of the obtained protected cyclic peptide.

Step (C) may further include (C-1) a step of producing a linear peptide in which the C-terminal of the linear peptide is or is not protected by a protecting group, an amino group of a side chain of a constituent amino acid is or is not protected, and a side chain of the constituent amino acid cysteine or cysteine derivative is modified with an alkylene group having a carboxy group by modifying, with an alkylene group having a carboxy group, a side chain of constituent amino acid cysteine or cysteine derivative of a linear peptide in which the C-terminal of the linear peptide is or is not protected by a protecting group, an amino group of a side chain of a constituent amino acid is or is not protected, and the side chain of the cysteine or cysteine derivative is not protected.

Other features of the invention will become apparent in the course of the following descriptions of exemplary embodiments which are given for illustration of the invention and are not intended to be limiting thereof.

### EXAMPLES

The reagents, apparatuses and materials used in the present invention are commercially available unless otherwise specified. In the present specification, when amino acid and the like are indicated by abbreviations, each indication is based on the abbreviation of the IUPAC-IUB Commission on Biochemical Nomenclature or conventional abbreviations in the art.

### Production Example 1

### Linear Peptide A (Completely-Protected Product)

Using Fmoc-Gly-OH, Fmoc-Leu-OH, Fmoc-Pro-OH, Fmoc-Cys(Mmt)-OH, Fmoc-Asn(Trt)-OH, Fmoc-Gln(Trt)-OH, Fmoc-Ile-OH, Fmoc-Tyr(Me)-OH, and chlorobutyric acid as starting materials, and (4,4′-bishydrophytyloxy)benzhydrylamine (denoted as NH₂-Dpm(4,4′-OPhy)) as a pseudo-solid-phase protecting group, a linear peptide A (completely-protected product) having the following sequence was synthesized according to a conventional method (see WO 2012/029794, and Angew Chem. Int. Ed. 2017. 27, (56), 7803, which are incorporated herein by reference in their entireties).

### Linear Peptide A (Completely-Protected Product)

Cl—C₃H₆CO-Tyr(Me)-Ile-Gln(Trt)-Asn(Trt)-Cys(Mmt)-Pro-Leu-Gly-NH -Dpm(4,4′-OPhy)

### Production Example 2

### Linear Peptide B (Completely-Protected Product)

Using Fmoc-Gly-OH, Fmoc-Leu-OH, Fmoc-Pro-OH, Fmoc-Cys(Mmt)-OH, Fmoc-Asn(Trt)-OH, Fmoc-Gln(Trt)-OH, Fmoc-Ile-OH, Fmoc-Tyr(Me)-OH, and chloroacetic acid as starting materials, and (4,4′-bishydrophytyloxy)benzhydrylamine (denoted as NH₂-Dpm(4,4′-OPhy)) as a pseudo solid phase protecting group, linear peptide B (completely-protected product) having the following sequence was synthesized according to a conventional method (see WO 2012/029794, and Angew Chem. Int. Ed. 2017. 27, (56), 7803, which are incorporated herein by reference in their entireties).

### Linear Peptide B (Completely-Protected Product)

### Cl—CH₂CO-Tyr(Me)-Ile-Gln(Trt)-Asn(Trt)-Cys(Mmt)-Pro-Leu-Gly-NH-Dpm(4,4′-OPhy)

### Production Example 3

### Linear Peptide C (Completely-Protected Product)

Using 3-mercapto(Trt)propionic acid, Fmoc-^{D}-Tyr(Et)-OH, Fmoc-Ile-OH, Fmoc-Thr(tBu)-OH, Fmoc-Asn(Trt)-OH, Fmoc-Cys(Trt)-OH, Fmoc-Pro-OH, Fmoc-Orn(Boc)-OH, and Fmoc-Gly-OH as starting materials, and (4,4′-bishydrophytyloxy)benzhydrylamine (denoted as NH₂-Dpm(4,4′-OPhy)) as a pseudo solid phase protecting group, linear peptide C (completely-protected product) having the following sequence was synthesized according to a conventional method (see WO 2012/029794, and Angew Chem. Int. Ed. 2017. 27, (56), 7803, which are incorporated herein by reference in their entireties).

### Linear Peptide C (Completely-Protected Product)

3-mercapto(Trt)propionyl-^{D}-Tyr(Et)-Ile-Thr(tBu)-Asn(Trt)-Cys(Trt) -Pro-Orn(Boc)-Gly-NH-Dpm(OPhy)

### Production Example 4

### Linear Peptide D (Completely-Protected Product)

Using Fmoc-Val-OH, Fmoc-^{D}-Phe-OH, Fmoc-Asp(OtBu)-OH, Fmoc-Gly-OH, and Fmoc-Arg(Pbf)-OH as starting materials, and 2-(3′-4′-5′-tri(2″,3″-dihydrophytyloxy)benzyloxy)-4-methoxybenzyl alcohol (denoted as HO-MTB(OPhy)) as a pseudo solid phase protecting group, linear peptide D (completely-protected product) having the following sequence was synthesized according to a conventional method (see WO 2012/029794, and Angew Chem. Int. Ed. 2017. 27, (56), 7803, which are incorporated herein by reference in their entireties).

### Linear Peptide D (Completely-Protected Product)

Fmoc-Arg(Pbf)-Gly-Asp(OtBu)-^{D}-Phe-Val-Arg(Pbf)-Gly-Asp(OtBu)-^{D}-Phe -Val-O-MTB(OPhy)

### Production Example 5

### Linear Peptide E (Protected Product in Which Only N-Terminal is Free)

Using Fmoc-Gly-OH, Fmoc-Leu-OH, Fmoc-Pro-OH, Fmoc-Cys(Mmt)-OH, Fmoc-Asn(Trt)-OH, Fmoc-Gln(Trt)-OH, Fmoc-Ile-OH, and Emoc-Tyr(Me)-OH as starting materials, and (4,4′-bishydrophytyloxy)benzhydrylamine (denoted as NH₂-Dpm(4,4′-OPhy)) as a pseudo solid phase protecting group, linear peptide E (protected product in which only N-terminal is free) having the following sequence was synthesized according to a conventional method (see WO 2012/029794, and Angew Chem. Int. Ed. 2017. 27, (56), 7803, which are incorporated herein by reference in their entireties).

### Linear Peptide E (Protected Product in Which Only N-Terminal is Free)

### H-Tyr(Me)-Ile-Gln(Trt)-Asn(Trt)-Cys(Mmt)-Pro-Leu-Gly-NH-Dpm(4,4′-OPhy)

### HPLC Measurement Method

The compounds obtained in the respective Examples mentioned below were subjected to HPLC measurement under the following conditions.
column: YMC-Pack ODS-AM 150×4.6 mm I.D.
S-5 μm 12 nm
mobile phase A: 0.05% TFA (trifluoroacetic acid)-containing water
mobile phase B: 0.05% TFA-containing acetonitrile
temperature: 40° C.
flow rate: 1.0 mL/min
detection wavelength: 220 nm
time program (mobile phase A ratio): 0-25 min 80%→20% 25-30 min 1%

### Method of Pre-Treatment Before HPLC Measurement

A pre-treatment for removing the side chain protecting group and the pseudo solid phase protecting group was performed before the above-mentioned HPLC measurement as necessary. The pre-treatment method is shown below.

A mixed solution of TFA/water/TIPS (triisopropylsilane)=95.0/2.5/2.5 (0.2 ml) was added to a part of the compounds obtained in the following respective Examples, and the mixture was stirred for 1-3 hr for deprotection. Then, 0.8 ml of 50% acetonitrile water was added and the solution was used as a sample for HPLC.

### Example 1

To linear peptide A (completely-protected product) (500 mg) synthesized in Production Example 1, and having a chlorobutyryl group at the N-terminal of the linear peptide and a methoxytrityl protecting group at the Cys residue in the peptide chain were added 5.0 ml of chloroform, 0.9 ml of trifluoroacetic acid, and 10 equivalents of mercaptopropionic acid, and the methoxytrityl group alone was removed in an ice bath. After stirring for 30 min, the reaction mixture was neutralized by adding 0.98 equivalents of pyridine, and partitioned by adding pure water in the same amount as chloroform. The obtained organic layer was concentrated using an evaporator, 5.0 ml of acetonitrile was added, and the precipitate was collected by filtration and dried to obtain a deprotected product (307 mg).

To the obtained deprotected product (102 mg) were added 4.1 ml of chloroform and 1 equivalent of DBU (1,8-diazabicyclo[5.4.0]-7-undecene), and cyclization between the terminal chlorobutyryl group and the SH group was performed. After stirring overnight, 20% aqueous NaCl (sodium chloride) solution (4.0 ml) was added, and the mixture was partitioned, concentrated, and dried to obtain cyclic peptide A (100 mg). To the obtained cyclic peptide A (100 mg) was added 0.3 ml of chloroform, and the mixture was stirred well. Thereafter, 2.7 ml of acetonitrile was added, the mixture was stirred well, and the insoluble material was filtered off. To the obtained insoluble material was added 0.2 ml of chloroform, and the mixture was stirred well. Thereafter, 1.4 ml of acetonitrile was added, the mixture was stirred well, and the insoluble material was filtered off again. This was repeated twice, and the obtained mother liquors were mixed and analyzed by HPLC, and it was confirmed that the HPLC purity of cyclic peptide A was improved from 74% to 89%. (yield 85% vs cyclic peptide A before filtration of insoluble material)

TOF-MS:m/z[M+H]⁺ 988.3

### Example 2

To linear peptide B (completely-protected product) (1.0 g) synthesized in Production Example 2, and having a chloroacetyl group at the N-terminal of the linear peptide and a methoxytrityl protecting group at the Cys residue in the peptide chain were added 10.0 ml of chloroform, 0.5 ml of trifluoroacetic acid, and 10 equivalents of mercaptopropionic acid, and the methoxytrityl group alone was removed in an ice bath. After stirring for 30 min, the reaction mixture was neutralized by adding 0.98 equivalents of pyridine, and partitioned by adding pure water in the same amount as chloroform. The obtained organic layer was concentrated using an evaporator, 5.0 ml of acetonitrile was added, and the precipitate was collected by filtration and dried to obtain a deprotected product (780 mg).

To the obtained deprotected product (195 mg) were added 2.0 ml of chloroform and 1 equivalent of DBU, and cyclization between the terminal chloroacetyl group and the SH group was performed. After stirring for 1 hr, the mixture was neutralized with 0.9 equivalents of methanesulfonic acid. 20% aqueous NaCl solution (2.0 ml) was added, and the mixture was partitioned, concentrated, and dried to obtain cyclic peptide B (206 mg). To the obtained cyclic peptide B was added 0.4 ml of chloroform, and the mixture was stirred well. Thereafter, 4.3 ml of acetonitrile was added, the mixture was stirred well, and the insoluble material was filtered off. Thereafter, concentration and drying were performed to obtain cyclic peptide B (147 mg). To the obtained insoluble material was added 0.3 ml of chloroform, and the mixture was stirred well. Thereafter, 3.2 ml of acetonitrile was added, the mixture was stirred well, and the insoluble material was filtered off again. The obtained mother liquors were mixed and analyzed by HPLC, and it was confirmed that the HPLC purity of cyclic peptide B was improved from 85% to 94%. (yield 99% vs cyclic peptide B before filtration of insoluble material)

TOF-MS:m/z[M+H]⁺ 960.7

### Example 3

To linear peptide C (completely-protected product) (200 mg) synthesized in Production Example 3, and having a mercaptopropionyl group protected by a trityl group at the N-terminal of the linear peptide and a trityl protecting group at the Cys residue were added 6.8 ml of CPME (cyclopentyl methyl ether), 1.2 ml of methanol, and 1 equivalent of iodine, and cyclization between SH groups was performed at room temperature. After stirring for 4 hr, the mixture was partitioned twice with an aqueous solution of ascorbic acid (133 mg) in water (8 ml), and washed twice with 20% aqueous NaCl solution. The obtained organic layer was concentrated using an evaporator and dried to obtain cyclic peptide C (195 mg). To the obtained cyclic peptide C (98 mg) was added 0.6 ml of chloroform, and the mixture was stirred well. Thereafter, 3.0 ml of acetonitrile was added, the mixture was stirred well, and the insoluble material was filtered off. To the obtained insoluble material was added 0.6 ml of chloroform, and the mixture was stirred well. Thereafter, 3.0 ml of acetonitrile was added, the mixture was stirred well, and the insoluble material was filtered off again. The obtained mother liquors were mixed and analyzed by HPLC, and it was confirmed that the HPLC purity of cyclic peptide C was improved from 67% to 87%. (yield 96% vs cyclic peptide C before filtration of insoluble material)

TOF-MS:m/z[M+H]⁺ 994.2

### Example 4

To linear peptide D (completely-protected product) (1.20 g) synthesized in Production Example 4, in which the N-terminal of the linear peptide is protected by an Fmoc group and the C-terminal is protected by a pseudo solid phase protecting group was added 24.0 ml of chloroform. In an ice bath, 3 equivalents of thiomalic acid and 11 equivalents of DBU were added, and only the Fmoc group at the N-terminal was removed under room temperature conditions. After stirring for 2 hr, a mixed solution of acetic acid (112 mg) and chloroform (0.6 ml) was added in an ice bath. Thereafter, the mixture was partitioned twice with an aqueous mixed solution of DMF (2.6 ml) and 5% aqueous sodium carbonate solution (10.6 ml), partitioned twice with DMF (3.8 ml) and 20% aqueous NaCl solution (5.8 ml), and washed once by partitioning with 20% aqueous NaCl solution (24 ml). The obtained organic layer was concentrated using an evaporator, and dried to obtain a solid (1.07 g). To the obtained solid was added 10.7 ml of HFIP (hexafluoroisopropanol), and only the pseudo solid phase protecting group at the C-terminal was removed at room temperature. After stirring for 5 hr, the mixture was concentrated using an evaporator. Thereafter, 64 ml of cyclohexane was added, and the solid was isolated and dried to obtain N-terminal non-protected, C-terminal non-protected linear peptide D (0.65 g).

To the above-mentioned linear peptide D (150 mg) were added 1.5 ml of chloroform, 0.5 equivalents of HOBt (1-hydroxybenzotriazole), and 1.1 equivalents of EDC.HCl (N-ethyl-N′-3-dimethylaminopropylcarbodiimide hydrochloride), and cyclization by intramolecular amide bond (lactam bond) was performed at room temperature. After stirring for 4 hr, the mixture was washed once with 20% aqueous NaCl solution (1.5 ml). The obtained organic layer was concentrated using an evaporator and dried to obtain cyclic peptide D (142 mg). To the obtained cyclic peptide D (142 mg) was added 1.7 ml of chloroform, and the mixture was stirred well. Thereafter, 4.0 ml of IPE (diisopropyl ether) was added, the mixture was stirred well, and the insoluble material was filtered off. To the obtained insoluble material was added 1.7 ml of chloroform, and the mixture was stirred well. Thereafter, 4.0 ml of IPE was added, the mixture was stirred well, and the insoluble material was filtered off again. This was repeated three times. The obtained mother liquors were mixed and analyzed by HPLC, and it was confirmed that the HPLC purity of cyclic peptide D was improved from 35% to 75%. (yield 86% vs cyclic peptide D before filtration of insoluble material)

TOF-MS:m/z[M+H]⁺ 1149.4

### Example 5

To linear peptide E (protected product in which only N-terminal is free) (1.57 g) synthesized in Production Example 5, and having unprotected N-terminal and a methoxytrityl protecting group at the Cys residue in the peptide chain were added 15.0 ml of chloroform, 7.4 ml of trifluoroacetic acid, and 10 equivalents of mercaptopropionic acid, and the methoxytrityl group alone was removed in an ice bath. After stirring for 7 hr, the reaction mixture was neutralized by adding 0.98 equivalents of pyridine, and partitioned by adding pure water in the same amount as chloroform. The obtained organic layer was concentrated using an evaporator, 15.7 ml of acetonitrile was added and the precipitate was collected by filtration and dried to obtain a deprotected product (1.04 g). To the obtained deprotected product (100 mg) was added 1.0 ml of chloroform, and 2.1 equivalents of chloroacetic acid and 6.0 equivalents of DBU were added in an ice bath to perform a nucleophilic substitution reaction of chloroacetic acid with the SH group in the Cys residue of the linear peptide under room temperature conditions. After stirring for 4 hr, the mixture was washed twice by partitioning with 20% aqueous NaCl solution (1.0 ml). The obtained organic layer was concentrated using an evaporator and dried to obtain an oil substance. To the obtained oil substance were added 4.1 ml of chloroform, 2.5 equivalents of HOBt, and 1.1 equivalents of EDC.HCl, and cyclization between the N-terminal amino group and the side chain carboxyl group was performed at room temperature. After stirring for 17 hr, the mixture was washed twice with 20% aqueous NaCl solution (4.1 ml). The obtained organic layer was concentrated using an evaporator and dried to obtain cyclic peptide E. To the obtained cyclic peptide E was added 0.6 ml of chloroform, and the mixture was stirred well. Thereafter, 6.9 ml of acetonitrile was added, the mixture was stirred well, and the insoluble material was filtered off. To the obtained insoluble material was added 0.1 ml of chloroform, and the mixture was stirred well. Thereafter, 1.6 ml of acetonitrile was added, the mixture was stirred well, and the insoluble material was filtered off again. The obtained mother liquors were mixed and analyzed by HPLC, and it was confirmed that the HPLC purity of cyclic peptide E was improved from 73% to 83%. (yield 89% vs cyclic peptide E before filtration of insoluble material)

TOF-MS:m/z[M+H]⁺ 960.7

### Example 6

To cyclic peptide A (388 mg) before filtration of insoluble material and synthesized in Example 1 were added a mixed solution (7.8 ml) of TFA (trifluoroacetic acid)/water/TIPS (triisopropylsilane)=95.0/2.5/2.5, and 10 equivalents of mercaptopropionic acid for final deprotection of all protecting groups. After stirring for 2 hr, IPE (diisopropyl ether) (38.8 ml) was added and the precipitate was collected by filtration and dried to obtain cyclic peptide A′ (unprotected product) (157 mg). To the obtained cyclic peptide A′ (5.0 mg) was added 30.0 μl of DMF, and the mixture was stirred well. Thereafter, 60.0 μl of water was added, the mixture was stirred well, and the insoluble material was filtered off. The obtained insoluble material was washed with a mixed solution of 100.0 μl of DMF and 200.0 μl of water. The obtained mother liquors were mixed and analyzed by HPLC, and it was confirmed that the HPLC purity of cyclic peptide A′ was improved from 66% to 88%. (yield 81% vs cyclic peptide A′ before filtration of insoluble material)

TOF-MS:m/z[M+H]⁺ 988.3

### Example 7

To cyclic peptide C (98 mg) before filtration of insoluble material and synthesized in Example 3 were added a mixed solution (2.0 ml) of TFA/water =97.5/2.5, and 10 equivalents of p-cresol for final deprotection of all protecting groups. After stirring for 16 hr, IPE (10.0 ml) was added and the precipitate was collected by filtration and dried to obtain cyclic peptide C′ (unprotected product) (49 mg). To the obtained cyclic peptide C′ (5.0 mg) was added 45.0 μl of methanol, and the mixture was stirred well. Thereafter, 45.0 μl of IPE was added, the mixture was stirred well, and the insoluble material was filtered off. The obtained insoluble material was washed with a mixed solution of 250.0 μl of methanol and 250.0 μl of IPE. The obtained mother liquors were mixed and analyzed by HPLC, and it was confirmed that the HPLC purity of cyclic peptide C′ was improved from 75% to 89%. (yield 91% vs cyclic peptide C′ before filtration of insoluble material)

TOF-MS:m/z[M+H]⁺ 994.2

### Example 8

To cyclic peptide D (128 mg) before filtration of insoluble material and synthesized in Example 4 was added a mixed solution (2.6 ml) of TFA/water/TIPS=95.0/2.5/2.5 for final deprotection of all protecting groups. After stirring for 3 hr, IPE (13.0 ml) was added and the precipitate was collected by filtration and dried to obtain cyclic peptide D′ (unprotected product) (79 mg). To the obtained cyclic peptide D′ (10.0 mg) was added 60.0 μl of DMF, and the mixture was stirred well. Thereafter, 240.0 μl of water was added, the mixture was stirred well, and the insoluble material was filtered off. The obtained insoluble material was washed with a mixed solution of 40.0 μl of DMF and 160.0 μl of water. The obtained mother liquors were mixed and analyzed by HPLC, and it was confirmed that the HPLC purity of cyclic peptide D′ was improved from 47% to 68%. (yield 76% vs cyclic peptide D′ before filtration of insoluble material)

TOF-MS:m/z[M+H]⁺ 1149.4

The above-mentioned Example 1 is an example in which cyclic peptide A (C—S type) is produced in Embodiment 1 of the present application by using chloroform as a good solvent and acetonitrile as a poor solvent. The above-mentioned Example 1 corresponds to Embodiment A.

The above-mentioned Example 2 is an example in which cyclic peptide B (C—S type) is produced in Embodiment 1 of the present application by using chloroform as a good solvent and acetonitrile as a poor solvent. The above-mentioned Example 2 corresponds to Embodiment A.

The above-mentioned Example 3 is an example in which cyclic peptide C (S—S type) is produced in Embodiment 1 of the present application by using chloroform as a good solvent and acetonitrile as a poor solvent.

The above-mentioned Example 4 is an example in which cyclic peptide D (lactam type) is produced in Embodiment 2 of the present application by using chloroform as a good solvent and IPE (diisopropyl ether) as a poor solvent.

The above-mentioned Example 5 is an example in which cyclic peptide E (C—S type) is produced in Embodiment 1 of the present application by using chloroform as a good solvent and acetonitrile as a poor solvent. The above-mentioned Example 5 corresponds to Embodiment B.

The above-mentioned Example 6 is an example in which cyclic peptide A (C—S type) is produced in Embodiment 1 of the present application by using DMF as a good solvent and water as a poor solvent. The above-mentioned Example 6 corresponds to Embodiment A.

The above-mentioned Example 7 is an example in which cyclic peptide C (S—S type) is produced in Embodiment 1 of the present application by using methanol as a good solvent and IPE as a poor solvent.

The above-mentioned Example 8 is an example in which cyclic peptide D (lactam type) is produced in Embodiment 2 of the present application by using DMF as a good solvent and water as a poor solvent.

### Example 9

To linear peptide E (protected product in which only N-terminal is free) (1.57 g) synthesized in Production Example 5, and having unprotected N-terminal and a methoxytrityl protecting group at the Cys residue in the peptide chain were added 15.0 ml of chloroform, 7.4 ml of trifluoroacetic acid, and 10 equivalents of mercaptopropionic acid, and the methoxytrityl group alone was removed in an ice bath. After stirring for 7 hr, the reaction mixture was neutralized by adding 0.98 equivalents of pyridine, and partitioned by adding pure water in the same amount as chloroform. The obtained organic layer was concentrated using an evaporator, 15.7 ml of acetonitrile was added and the precipitate was collected by filtration and dried to obtain a deprotected product (1.04 g). To the obtained deprotected product (875 mg) was added 8.8 ml of chloroform, and 2.1 equivalents of chloroacetic acid and 6.0 equivalents of DBU were added in an ice bath to perform a nucleophilic substitution reaction of chloroacetic acid with the SH group in the Cys residue of the linear peptide under room temperature conditions. After stirring for 2 hr, the mixture was neutralized by adding a mixture of 4.5 equivalents of acetic acid and 0.5 ml of chloroform, and washed once by partitioning with 20% aqueous NaCl solution (8.8 ml). To the obtained organic layer were added 0.5 equivalents of HOBt and 1.6 equivalents of EDC.HCl, and cyclization between the N-terminal amino group and the side chain carboxyl group was performed at room temperature. After stirring for 18 hr, the mixture was washed once with 20% aqueous NaCl solution (10.0 ml). The obtained organic layer was concentrated using an evaporator and dried to obtain a solid. To the obtained solid were added a mixed solution (9.5 ml) of TFA/water/TIPS=95.0/2.5/2.5 and 10 equivalents of mercaptopropionic acid for final deprotection of all protecting groups. After stirring for 20 hr, IPE (47.0 ml) was added, and the precipitate was collected by filtration and dried to obtain 375 mg of cyclic peptide E′ (completely-unprotected product) before filtration of insoluble material. To the obtained cyclic peptide E′ (152.0 mg) before filtration of insoluble material was added 12.1 ml of methanol, and the mixture was stirred well. Thereafter, 18.2 ml of IPE was successively added, the mixture was stirred well, and the insoluble material was filtered off. To the obtained insoluble material was added 12.1 ml of methanol, and the mixture was stirred well. Thereafter, 18.2 ml of IPE was successively added, the mixture was stirred well, and the insoluble material was filtered off again. The obtained mother liquors were mixed and quantitatively analyzed by HPLC, and it was confirmed that the HPLC purity of cyclic peptide E′ was improved from 47% to 86%. (yield 90% vs cyclic peptide E′ before filtration of insoluble material)

TOF-MS:m/z[M+H]⁺ 960.5

### Production Example 6

### Linear Peptide F (N-Terminal Boc Completely-Protected Product)

Using Boc-Cys(Trt)-OH, Fmoc-Tyr(tBu)-OH, Fmoc-Ile-OH, Fmoc-Gln(Trt)-OH, Fmoc-Asn(Trt)-OH, Fmoc-Cys(Trt)-OH, Fmoc-Pro-OH, Fmoc-Leu-OH, Fmoc-Gly-OH as starting materials, and Siber Amide resin as a solid phase protecting group, linear peptide F (N-terminal Boc completely-protected product) having the following sequence was synthesized according to a conventional method.

### Linear Peptide F (N-Terminal Boc Completely-Protected Product)

Boc-Cys(Trt)-Tyr(tBu)-Ile-Gln(Trt)-Asn(Trt)-Cys(Trt)-Pro-Leu-Gly-NH-Siber resin

### Example 10

To linear peptide F (N-terminal Boc completely-protected product) (632 mg) synthesized in Production Example 6, and having a Boc protecting group at the N-terminal were added 6.3 ml of chloroform and 126 μl of trifluoroacetic acid, and the solid phase protecting group alone was removed under room temperature conditions. After stirring for 1 hr, the deprotected solid phase protecting group was filtered to obtain a filtrate. To the filtered solid phase protecting group were added 6.3 ml of chloroform and 126 μl of trifluoroacetic acid again. After stirring for 1 hr under room temperature conditions, the deprotected solid phase protecting group was filtered to obtain a filtrate. The obtained filtrates were mixed and 147 μl of piperidine was added in an ice bath to neutralize the mixture, and the mixture was concentrated using an evaporator. In an ice bath, 25.2 ml of IPE was added and the precipitate was collected by filtration and dried to obtain protected peptide amide product (697 mg). To the obtained protected peptide amide product (300 mg) were added 12.8 ml of chloroform, 2.3 ml of methanol, and 3.0 equivalents of iodine, and cyclization between the SH groups was performed at room temperature. After stirring for 30 min, the mixture was partitioned twice with an aqueous solution of ascorbic acid (148.6 mg) in water (5.0 ml), and washed with 20% aqueous NaCl (sodium chloride) solution. The obtained organic layer was concentrated using an evaporator, 10.0 ml of IPE was added in an ice bath, and the precipitate was collected by filtration and dried to obtain 177 mg of cyclic peptide F before filtration of insoluble material. To the obtained cyclic peptide F (100.3 mg) before filtration of insoluble material was added 2.2 ml of THF, and the mixture was stirred well. Thereafter, 2.8 ml of hexane was successively added, the mixture was stirred well, and the insoluble material was filtered off. To the obtained insoluble material was added 2.2 ml of THF, and the mixture was stirred well. Thereafter, 2.8 ml of hexane was successively added, the mixture was stirred well, and the insoluble material was filtered off again. The obtained mother liquors were mixed and quantitatively analyzed by HPLC, and it was confirmed that the HPLC purity of cyclic peptide F was improved from 49% to 70%. (yield 78% vs cyclic peptide F before filtration of insoluble material)

TOF-MS:m/z[M+H]⁺ 1007.4

### Example 11

To linear peptide F (N-terminal Boc completely-protected product) (556 mg) synthesized in Production Example 6, and having a Boc protecting group at the N-terminal were added 5.6 ml of chloroform and 111 μl of trifluoroacetic acid, and the solid phase protecting group alone was removed under room temperature conditions. After stirring for 1 hr, the mixture was neutralized by adding 111 μl of piperidine in an ice bath, the deprotected solid phase protecting group was filtered to obtain a filtrate. To the filtered solid phase protecting group were added 5.6 ml of chloroform and 111 μl of trifluoroacetic acid again. After stirring for 1 hr under room temperature conditions, the mixture was neutralized by adding 111 μl of piperidine in an ice bath, the deprotected solid phase protecting group was filtered to obtain a filtrate. The obtained filtrates were mixed, concentrated using an evaporator and dried to obtain a protected peptide amide product. To the obtained protected peptide amide product were added 13.1 ml of chloroform, 2.5 ml of methanol, and 3.0 equivalents of iodine, and cyclization between the SH groups was performed at room temperature. After stirring for 30 min, the mixture was partitioned twice with an aqueous solution of ascorbic acid (152.2 mg) in water (5.0 ml), and washed with 20% aqueous NaCl solution (10.0 ml). The obtained organic layer was concentrated using an evaporator, 10.0 ml of IPE was added in an ice bath, and the precipitate was collected by filtration and dried to obtain cyclic peptide F′ (163 mg). To the obtained cyclic peptide F′ (163 mg) was added a mixed solution (3.3 ml) of TFA/water/p-cresol=97.5/2.5/2.5 for final deprotection of all protecting groups. After stirring for 3 hr, IPE (9.5 ml) was added in an ice bath, and the precipitate was collected by filtration and dried to obtain 82 mg of cyclic peptide F″ (completely-unprotected product) before filtration of insoluble material.

To the obtained cyclic peptide F″ (63 mg) before filtration of insoluble material was added 1.3 ml of methanol, and the mixture was stirred well. Thereafter, 1.9 ml of IPE was successively added, the mixture was stirred well, and the insoluble material was filtered off. To the obtained insoluble material was added 0.6 ml of methanol, and the mixture was stirred well. Thereafter, 0.9 ml of IPE was successively added, the mixture was stirred well, and the insoluble material was filtered off again. The obtained mother liquors were mixed and quantitatively analyzed by HPLC, and it was confirmed that the HPLC purity of cyclic peptide F″ was improved from 43% to 81%. (yield 97% vs cyclic peptide F before filtration of insoluble material)

TOF-MS:m/z[M+H]⁺ 1007.3

### Production Example 7

### Linear Peptide G (N-Terminal Ac Completely-Protected Product)

Using Fmoc-Lys(Mtt)-OH, Fmoc-Tyr(tBu)-OH, Fmoc-Ile-OH, Fmoc-Pro-OH, Fmoc-Gln(Trt)-OH, Fmoc-Ala-OH, Fmoc-Glu(OPis)-OH, and Fmoc-Leu-OH as starting materials, and (4,4′-bishydrophytyloxy)benzhydrylamine (denoted as NH₂-Dpm(4,4′-OPhy)) as a pseudo solid phase protecting group, linear peptide G (N-terminal Ac completely-protected product) having the following sequence was synthesized according to a conventional method (see WO 2012/029794, and Angew Chem. Int. Ed. 2017. 27, (56), 7803). The N-terminal Ac group of the following peptide was acetylated using acetic anhydride and N-ethyldiisopropylamine.

### Linear Peptide G (N-Terminal Ac Completely-Protected Product)

### Ac-Lys(Mtt)-Tyr(tBu)-Ile-Pro-Gln(Trt)-Ala-Ala-Gln(Trt)-Ala-Ala-Glu(OPis) -Ile-Pro-Leu-NH-Dpm(4,4′-OPhy)

### Example 12

To linear peptide G (N-terminal Ac completely-protected product) (489 mg) synthesized in Production Example 7, and having an Ac protecting group at the N-terminal, a methyltrityl protecting group at the Lys residue in the peptide chain, and a 2-phenylisopropyl protecting group at the Glu residue in the peptide chain were added 14.8 ml of chloroform and 225 μl of trifluoroacetic acid, and the methyltrityl group and the 2-phenylisopropyl group were removed under room temperature conditions. After stirring for 2 hr, the mixture was neutralized by adding 237 μl of piperidine in an ice bath, and partitioned twice with 10.0 ml of 5% aqueous Na₂CO₃ (sodium carbonate) solution and washed twice with 10.0 ml of 20% aqueous NaCl (sodium chloride) solution. The obtained organic layer was concentrated using an evaporator, 10.0 ml of acetonitrile was added, and the precipitate was collected by filtration and dried to obtain a deprotected product G (410 mg). To the obtained deprotected product G (410 mg) were added 25.0 ml of DMF, 1.0 equivalent of HOBt, and 3.0 equivalents of EDC.HCl, and cyclization by an intramolecular amide bond (lactam bond) was performed at room temperature. After stirring overnight, the mixture was partitioned with CPME (50.0 ml) and 20% aqueous NaCl solution (50.0 ml). The obtained organic layer was concentrated using an evaporator, 10.0 ml of acetonitrile was added, and the precipitate was collected by filtration and dried to obtain cyclic peptide G (426 mg) before filtration of insoluble material.

To the obtained cyclic peptide G (198 mg) before filtration of insoluble material was added 2.3 ml of chloroform, and the mixture was stirred well. Thereafter, 57.2 ml of acetonitrile was successively added, the mixture was stirred well, and the insoluble material was filtered off. To the obtained insoluble material was added 1.2 ml of chloroform, and the mixture was stirred well. Thereafter, 58.3 ml of acetonitrile was successively added, the mixture was stirred well, and the insoluble material was filtered off again. The obtained mother liquors were mixed and quantitatively analyzed by HPLC, and it was confirmed that the HPLC purity of cyclic peptide F was improved from 40% to 71%. (yield 90% vs cyclic peptide G before filtration of insoluble material)

TOF-MS:m/z[M+H]⁺ 1535.8

### Production Example 8

### Linear Peptide H (Completely-Protected Product)

Using Fmoc-Gly-OH, Fmoc-Leu-OH, Fmoc-Pro-OH, Fmoc-Cys(Mmt)-OH, Fmoc-Asn(Trt)-OH, Fmoc-Gln(Trt)-OH, Fmoc-Ile-OH, Fmoc-Tyr(tBu)-OH, and chloroacetic acid as starting materials, and Siber Amide resin as a solid phase protecting group, linear peptide H (completely-protected product) having the following sequence was synthesized according to a conventional method.

### Linear Peptide H (Completely-Protected Product)

Cl—CH₂CO-Tyr(tBu)-Ile-Gln(Trt)-Asn(Trt)-Cys(Mmt)-Pro-Leu-Gly-NH-Siber resin

### Example 13

To linear peptide H (completely-protected product) (658 mg) synthesized in Production Example 8, and having a chloroacetyl group at the N-terminal of the linear peptide and a methoxytrityl protecting group at the Cys residue in the peptide chain were added 12.5 ml of chloroform, 656 μl of trifluoroacetic acid, and 10 equivalents of mercaptopropionic acid, and the methoxytrityl group and the solid phase protecting group were removed in an ice bath. After stirring for 30 min, the reaction mixture was neutralized by adding 694 μl of pyridine. Thereafter, the deprotected solid phase protecting group was filtered to obtain a filtrate. To the filtered solid phase protecting group were added 12.5 ml of chloroform, 656 μl of trifluoroacetic acid, and 10 equivalents of mercaptopropionic acid again, and the methoxytrityl group and the solid phase protecting group were removed in an ice bath. After stirring for 30 min, the reaction mixture was neutralized by adding 694 μl of pyridine. Thereafter, the deprotected solid phase protecting group was filtered to obtain a filtrate. The obtained filtrates were mixed, and the mixture was partitioned by adding 13.2 ml of pure water. The obtained organic layer was concentrated using an evaporator, 32.9 ml of IPE was added in an ice bath, and the precipitate was collected by filtration and dried to obtain protected peptide amide product (203 mg) in which only the SH group of the Cys residue in the peptide chain was deprotected.

To the obtained protected peptide amide product (203 mg) in which only the SH group of the Cys residue in the peptide chain was deprotected were added 2.0 ml of chloroform and 1.5 equivalents of DBU, and cyclization between the terminal chloroacetyl group and the SH group was performed. After stirring for 30 min, the mixture was neutralized by adding 1.4 equivalents of acetic acid in an ice bath. Thereafter, 20% aqueous NaCl solution (8.0 ml) was added, and the mixture was partitioned twice, concentrated, and dried to obtain cyclic peptide H (199 mg). To the obtained cyclic peptide H (199 mg) was added 7.2 ml of chloroform, and the mixture was stirred well. Thereafter, 12.7 ml of IPE was successively added, the mixture was stirred well, and the insoluble material was filtered off. To the obtained insoluble material was added 7.2 ml of chloroform, and the mixture was stirred well. Thereafter, 12.7 ml of IPE was successively added, the mixture was stirred well, and the insoluble material was filtered off again. This was repeated 4 times and the obtained mother liquors were mixed and quantitatively analyzed by HPLC, and it was confirmed that the HPLC purity of cyclic peptide H was improved from 55% to 79%. (yield 70% vs cyclic peptide H before filtration of insoluble material)

TOF-MS:m/z[M+H]⁺ 946.4

### Production Example 9

### Linear Peptide I (Protected Product in Which Only N-Terminal is Free)

Using Fmoc-Gly-OH, Fmoc-Leu-OH, Fmoc-Pro-OH, Fmoc-Cys(Mmt)-OH, Fmoc-Asn(Trt)-OH, Fmoc-Gln(Trt)-OH, Fmoc-Ile-OH, and Fmoc-Tyr(tBu)-OH as starting materials, and Siber Amide resin as a solid phase protecting group, linear peptide F (protected product in which only N-terminal is free) having the following sequence was synthesized according to a conventional method.

### Linear Peptide I (Protected Product in Which Only N-Terminal is Free)

H-Tyr(tBu)-Ile-Gln(Trt)-Asn(Trt)-Cys(Mmt)-Pro-Leu-Gly-NH-Siber resin

### Example 14

To linear peptide I (protected product in which only N-terminal is free) (517 mg) synthesized in Production Example 9, and having an unprotected N-terminal, and a methoxytrityl protecting group at the Cys residue in the peptide chain were added 10.1 ml of chloroform, 0.5 ml of trifluoroacetic acid, and 10 equivalents of mercaptopropionic acid, and the methoxytrityl group and the solid phase protecting group were removed in an ice bath. After stirring for 30 min, the reaction mixture was neutralized by adding 533 μl of pyridine. Thereafter, the deprotected solid phase protecting group was filtered to obtain a filtrate. To the filtered solid phase protecting group were added 10.1 ml of chloroform, 0.5 ml of trifluoroacetic acid, and 10 equivalents of mercaptopropionic acid again, and the methoxytrityl group and the solid phase protecting group were removed in an ice bath. After stirring for 30 min, the reaction mixture was neutralized by adding 533 μl of pyridine. Thereafter, the deprotected solid phase protecting group was filtered to obtain a filtrate. The obtained filtrates were mixed, and the mixture was partitioned three times by adding 20.2 ml of pure water. The obtained organic layer was concentrated using an evaporator, 25.3 ml of IPE was added in an ice bath, and the precipitate was collected by filtration and dried to obtain a protected peptide amide product (188 mg) in which only the N-terminal and SH group at the Cys residue in the peptide chain were deprotected. To the obtained protected peptide amide product (188 mg) in which only the N-terminal and SH group at the Cys residue in the peptide chain were deprotected was added 1.9 ml of chloroform, 5.0 equivalents of chloroacetic acid and 9.0 equivalents of DBU were added in an ice bath to perform a nucleophilic substitution reaction of chloroacetic acid with the SH group at the Cys residue in the linear peptide under room temperature conditions. After stirring for 1 hr, the mixture was partitioned with 5% aqueous Na₂CO₃ solution (7.5 ml) and then washed twice by partitioning with 20% aqueous NaCl solution. To the obtained organic layer were added 20.0 ml of chloroform, 2.0 equivalents of HOBt, and 1.1 equivalents of EDC.HCl, and cyclization between the N-terminal amino group and the side chain carboxyl group was performed at room temperature. After stirring for 1 hr, the mixture was washed once with 20% aqueous NaCl solution (20.0 ml). The obtained organic layer was concentrated using an evaporator and dried to obtain cyclic peptide I′ (183 mg) before filtration of insoluble material.

To the obtained cyclic peptide I′ (101 mg) before filtration of insoluble material was added 3.7 ml of chloroform, and the mixture was stirred well. Thereafter, 6.4 ml of IPE was successively added, the mixture was stirred well, and the insoluble material was filtered off. To the obtained insoluble material was added 3.7 ml of chloroform, and the mixture was stirred well. Thereafter, 6.4 ml of IPE was successively added, the mixture was stirred well, and the insoluble material was filtered off again. This was repeated 4 times and the obtained mother liquors were mixed and quantitatively analyzed by HPLC, and it was confirmed that the HPLC purity of cyclic peptide I′ was improved from 38% to 70%. (yield 87% vs cyclic peptide I′

TOF-MS:m/z[M+H]⁺ 946.4

### Example 15

To linear peptide G (N-terminal Ac completely-protected product) (489 mg) synthesized in Production Example 7, and having an Ac protecting group at the N-terminal, a methyltrityl protecting group at the Lys residue in the peptide chain, and a 2-phenylisopropyl protecting group at the Glu residue in the peptide chain were added 14.3 ml of chloroform, 975 μl of trifluoroacetic acid, and 10 equivalents of mercaptopropionic acid, and the methyltrityl group and the 2-phenylisopropyl group were removed in an ice bath. After stirring for 2 hr, the reaction mixture was neutralized by adding 1.0 g of pyridine and partitioned twice with 15.0 ml of pure water in an ice bath. The obtained organic layer was concentrated using an evaporator, 10.0 ml of acetonitrile was added, and the precipitate was collected by filtration and dried to obtain a deprotected product G′ (404 mg). To the obtained deprotected product G′ (300 mg) were added 18.0 ml of DMF, 1.0 equivalent of HOBt, and 3.0 equivalents of EDC.HCl, and cyclization by an intramolecular amide bond (lactam bond) was performed at room temperature. After stirring overnight, the mixture was partitioned by adding CPME (36.0 ml) and 20% aqueous NaCl solution (36.0 ml). The obtained organic layer was concentrated using an evaporator, 10.0 ml of acetonitrile was added, and the precipitate was collected by filtration and dried to obtain cyclic peptide G′ (276 mg). To the obtained cyclic peptide G′ (276.0 mg) were added a mixed solution (5.5 ml) of TFA/water/TIPS=95.0/2.5/2.5 and 10 equivalents of mercaptopropionic acid to perform final deprotection to remove all protecting groups. After stirring for 17 hr, IPE (27.6 ml) was added and the precipitate was collected by filtration and dried to obtain cyclic peptide G″ (completely-unprotected product) (146 mg) before filtration of insoluble material.

To the obtained cyclic peptide G″ (58 mg) before filtration of insoluble material was added 0.9 ml of methanol, and the mixture was stirred well. Thereafter, 2.0 ml of IPE was successively added, the mixture was stirred well, and the insoluble material was filtered off. To the obtained insoluble material was added 0.9 ml of methanol, and the mixture was stirred well. Thereafter, 2.0 ml of IPE was successively added, the mixture was stirred well, and the insoluble material was filtered off again. This was repeated 3 times. The obtained mother liquors were mixed and quantitatively analyzed by HPLC, and it was confirmed that the HPLC purity of cyclic peptide G′ was improved from 40% to 70%. (yield 92% vs cyclic peptide G′ before filtration of insoluble material)

TOF-MS:m/z[M+H]⁺ 1535.7

### Production Example 10

### Linear Peptide J (Completely-Protected Product)

Using Fmoc-Leu-OH, Fmoc-Pro-OH, Fmoc-Ile-OH, Fmoc-Cys(Mmt)-OH, Fmoc-Ala-OH, Fmoc-Gln(Trt)-OH, Fmoc-Tyr(tBu)-OH, and chloroacetic acid as starting materials, and (4,4′-bishydrophytyloxy)benzhydrylamine (denoted as NH2-Dpm(4,4′-OPhy)) as a pseudo solid phase protecting group, linear peptide G (completely-protected product) having the following sequence was synthesized according to a conventional method (see WO 2012/029794, and Angew Chem. Int. Ed. 2017. 27, (56), 7803).

### Linear Peptide J (Completely-Protected Product)

### Cl—CH₂CO-Tyr(tBu)-Ile-Pro-Tyr(tBu)-Gln(Trt)-Ala-Ala-Cys(Mmt)-Ile -Pro-Leu-NH-Dpm(4,4′-OPhy)

### Example 16

To linear peptide J (completely-protected product) (1.50 g) synthesized in Production Example 10, and having a chloroacetyl group at the N-terminal of the linear peptide and a methoxytrityl protecting group at the Cys residue in the peptide chain were added 30.0 ml of chloroform, 525 μl of trifluoroacetic acid, and 10 equivalents of mercaptopropionic acid, and the methoxytrityl group alone was removed in an ice bath. After stirring for 30 min, the reaction mixture was neutralized by adding 536 μl of pyridine. Thereafter, 30.0 ml of pure water was added, and the mixture was partitioned twice. The obtained organic layer was concentrated using an evaporator, 30.0 ml of acetonitrile was added at room temperature, and the precipitate was collected by filtration and dried to obtain a deprotected product (1.11 g) in which only the SH group at the Cys residue in the peptide chain was deprotected.

To the obtained deprotected product (300 mg) in which only the SH group at the Cys residue in the peptide chain was deprotected were added 6.0 ml of chloroform and 1.5 equivalents of DBU and cyclization between the terminal chloroacetyl group and the SH group was performed. After stirring for 3 hr, the mixture was neutralized by adding 0.5 equivalents of acetic acid in an ice bath. Thereafter, 20% aqueous NaCl solution (6.0 ml) was added, and the mixture was partitioned twice. The obtained organic layer was concentrated using an evaporator, 30.0 ml of acetonitrile was added at room temperature, and the precipitate was collected by filtration and dried to obtain cyclic peptide J. To the obtained cyclic peptide J was added 2.4 ml of chloroform, and the mixture was stirred well. Thereafter, 48.1 ml of acetonitrile was successively added, the mixture was stirred well, and the insoluble material was filtered off. To the obtained insoluble material was added 0.9 ml of chloroform, and the mixture was stirred well. Thereafter, 18.9 ml of acetonitrile was successively added, the mixture was stirred well, and the insoluble material was filtered off again. The obtained mother liquors were mixed and quantitatively analyzed by HPLC, and it was confirmed that the HPLC purity of cyclic peptide H was improved from 51% to 88%. (yield 83% vs cyclic peptide J before filtration of insoluble material)

TOF-MS:m/z[M+H]⁺ 1290.6

### Production Example 11

### Linear Peptide K (Protected Product in Which Only N-Terminal is Free)

Using Fmoc-Leu-OH, Fmoc-Pro-OH, Fmoc-Ile-OH, Fmoc-Cys(Mmt)-OH, Fmoc-Ala-OH, Fmoc-Gln(Trt)-OH, and Fmoc-Tyr(tBu)-OH as starting materials, and (4,4′-bishydrophytyloxy)benzhydrylamine (denoted as NH₂-Dpm(4,4′-OPhy)) as a pseudo solid phase protecting group, linear peptide K (protected product in which only N-terminal is free) having the following sequence was synthesized according to a conventional method (see WO 2012/029794, and Angew Chem. Int. Ed. 2017. 27, (56), 7803).

### Linear Peptide K (Protected Product in Which Only N-Terminal is Free)

### H-Tyr(tBu)-Ile-Pro-Tyr(tBu)-Gln(Trt)-Ala-Ala-Cys(Mmt)-Ile-Pro-Leu -NH-Dpm(4,4′-OPhy)

### Example 17

To linear peptide K (protected product in which only N-terminal is free) (1.50 g) synthesized in Production Example 11, and having unprotected N-terminal and a methoxytrityl protecting group at the Cys residue in the peptide chain were added 30.0 ml of chloroform, 525 μl of trifluoroacetic acid, and 10 equivalents of mercaptopropionic acid, and the methoxytrityl group alone was removed in an ice bath. After stirring for 30 min, the reaction mixture was neutralized by adding 536 μl of pyridine. Thereafter, 30.0 ml of pure water was added, and the mixture was partitioned twice. The obtained organic layer was concentrated using an evaporator, 30.0 ml of acetonitrile was added at room temperature, and the precipitate was collected by filtration and dried to obtain deprotected product (1.11 g) in which only the SH group at the Cys residue in the peptide chain was deprotected. To the obtained deprotected product (500 mg) in which only the SH group at the Cys residue in the peptide chain was deprotected was added 5.0 ml of chloroform, and 5.0 equivalents of chloroacetic acid and 9.0 equivalents of DBU were added in an ice bath to perform a nucleophilic substitution reaction of chloroacetic acid with the SH group at the Cys residue in the linear peptide under room temperature conditions. After stirring for 1 hr, the mixture was partitioned twice with 5% aqueous Na₂CO₃ solution (5.0 ml) and then washed by partitioning with 20% aqueous NaCl solution. The obtained organic layer was concentrated using an evaporator, 15.0 ml of acetonitrile was added at room temperature, and the precipitate was collected by filtration and dried to obtain a solid. To the obtained solid were added 17.4 ml of chloroform, 2.0 equivalents of HOBt, and 2.0 equivalents of EDC.HCl, and cyclization between the N-terminal amino group and the side chain carboxyl group was performed at room temperature. After stirring for 1 hr, the mixture was washed once with 20% aqueous NaCl solution (20.0 ml). The obtained organic layer was concentrated using an evaporator and dried to obtain cyclic peptide K (322 mg) before filtration of insoluble material.

To the obtained cyclic peptide K (200 mg) before filtration of insoluble material was added 1.9 ml of chloroform, and the mixture was stirred well. Thereafter, 38.1 ml of acetonitrile was successively added, the mixture was stirred well, and the insoluble material was filtered off. To the obtained insoluble material was added 1.9 ml of chloroform, and the mixture was stirred well. Thereafter, 38.1 ml of acetonitrile was successively added, the mixture was stirred well, and the insoluble material was filtered off again. The obtained mother liquors were mixed and quantitatively analyzed by HPLC, and it was confirmed that the HPLC purity of cyclic peptide K was improved from 48% to 87%. (yield 95% vs cyclic peptide K before filtration of insoluble material)

TOF-MS:m/z[M+H]⁺ 1290.6

### Example 18

To linear peptide J (completely-protected product) (3.0 g) synthesized in Production Example 10, and having a chloroacetyl group at the N-terminal of the linear peptide and a methoxytrityl protecting group at the Cys residue in the peptide chain were added 60.0 ml of chloroform, 1.35 ml of trifluoroacetic acid, and 10 equivalents of mercaptopropionic acid, and the methoxytrityl group alone was removed in an ice bath. After stirring for 3 hr, the reaction mixture was neutralized by adding 1.4 ml of pyridine. Thereafter, 60.0 ml of pure water was added, and the mixture was partitioned twice. The obtained organic layer was concentrated using an evaporator, 60.0 ml of acetonitrile was added at room temperature, and the precipitate was collected by filtration and dried to obtain deprotected product (2.70 g) in which only the SH group at the Cys residue in the peptide chain was deprotected.

To the obtained deprotected product (794 mg) in which only the SH group at the Cys residue in the peptide chain was deprotected were added 15.9 ml of chloroform and 1.5 equivalents of DBU, and cyclization between the terminal chloroacetyl group and the SH group was performed. After stirring for 3 hr, the mixture was neutralized by adding 0.5 equivalents of acetic acid in an ice bath. Thereafter, 20% aqueous NaCl solution 50.0 ml was added, and the mixture was partitioned twice. The obtained organic layer was concentrated using an evaporator, 50.0 ml of acetonitrile was added at room temperature, and the precipitate was collected by filtration and dried to obtain cyclic peptide J′ (669 mg). To the obtained cyclic peptide J′ (669 mg) were added a mixed solution (12.7 ml) of TFA/water/TIPS=95.0/2.5/2.5 and 10 equivalents of mercaptopropionic acid to perform final deprotection to remove all protecting groups. After stirring for 6 hr, IPE (66.9 ml) was added and the precipitate was collected by filtration and dried to obtain cyclic peptide J″ (completely-unprotected product) (379 mg) before filtration of insoluble material.

To the obtained cyclic peptide J″ (201 mg) was added 28.7 ml of methanol, and the mixture was stirred well. Thereafter, 71.6 ml of IPE was successively added, the mixture was stirred well, and the insoluble material was filtered off. The obtained mother liquor was quantitatively analyzed by HPLC, and it was confirmed that the HPLC purity of cyclic peptide J″ was improved from 51% to 77%. (yield 93% vs cyclic peptide J″ before filtration of insoluble material)

TOF-MS:m/z[M+H]⁺ 1290.5

### Example 19

To linear peptide K (protected product in which only N-terminal is free) (1.50 g) synthesized in Production Example 11 and having unprotected N-terminal, and a methoxytrityl protecting group at the Cys residue in the peptide chain were added 30.0 ml of chloroform, 525 μl of trifluoroacetic acid, and 10 equivalents of mercaptopropionic acid, and the methoxytrityl group alone was removed in an ice bath. After stirring for 30 min, the reaction mixture was neutralized by adding 536 μl of pyridine. Thereafter, 30.0 ml of pure water was added, and the mixture was partitioned twice. The obtained organic layer was concentrated using an evaporator, 30.0 ml of acetonitrile was added at room temperature, and the precipitate was collected by filtration and dried to obtain a deprotected product (1.11 g) in which only the SH group at the Cys residue in the peptide chain was deprotected. To the obtained deprotected product (500 mg) in which only the SH group at the Cys residue in the peptide chain was deprotected was added 5.0 ml of chloroform, and 5.0 equivalents of chloroacetic acid and 9.0 equivalents of DBU were added in an ice bath to perform a nucleophilic substitution reaction of chloroacetic acid with the SH group at the Cys residue in the linear peptide under room temperature conditions. After stirring for 1 hr, the mixture was partitioned twice with 5% aqueous Na₂CO₃ solution (5.0 ml) and then washed by partitioning with 20% aqueous NaCl solution. The obtained organic layer was concentrated using an evaporator, 15.0 ml of acetonitrile was added at room temperature, and the precipitate was collected by filtration and dried to obtain a solid. To the obtained solid were added 17.4 ml of chloroform, 2.0 equivalents of HOBt, and 2.0 equivalents of EDC.HCl, and cyclization between the N-terminal amino group and the side chain carboxyl group was performed at room temperature. After stirring for 1 hr, the mixture was washed once with 20% aqueous NaCl solution (20.0 ml). The obtained organic layer was concentrated using an evaporator and dried to obtain cyclic peptide K′ (322 mg). To the obtained cyclic peptide K′ (285 mg) were added a mixed solution of TFA/water/TIPS=95.0/2.5/2.5 (5.7 ml) and 10 equivalents of mercaptopropionic acid to perform final deprotection to remove all protecting groups. After stirring for 5 hr, IPE (28.5 ml) was added and the precipitate was collected by filtration and dried to obtain cyclic peptide K″ (completely-unprotected product) (154 mg) before filtration of insoluble material.

To the obtained cyclic peptide K″ (101 mg) before filtration of insoluble material was added 8.6 ml of methanol, and the mixture was stirred well. Thereafter, 21.6 ml of IPE was successively added, the mixture was stirred well, and the insoluble material was filtered off. The obtained mother liquor was quantitatively analyzed by HPLC, and it was confirmed that the HPLC purity of cyclic peptide K″ was improved from 47% to 85%. (yield 87% vs cyclic peptide K″ before filtration of insoluble material)

TOF-MS:m/z[M+H]⁺ 1290.5

The above-mentioned Example 9 is an example in which cyclic peptide E (C—S type) is produced in Embodiment 1 of the present application by using methanol as a good solvent and IPE as a poor solvent. The above-mentioned Example 9 corresponds to Embodiment B.

The above-mentioned Example 10 is an example in which cyclic peptide F (S—S type) is produced in Embodiment 2 of the present application by using THE as a good solvent and hexane as a poor solvent.

The above-mentioned Example 11 is an example in which cyclic peptide F (S—S type) is produced in Embodiment 2 of the present application by using methanol as a good solvent and IPE as a poor solvent.

The above-mentioned Example 12 is an example in which cyclic peptide G (lactam type) is produced in Embodiment 1 of the present application by using chloroform as a good solvent and acetonitrile as a poor solvent.

The above-mentioned Example 13 is an example in which cyclic peptide H (C—S type) is produced in Embodiment 2 of the present application by using chloroform as a good solvent and IPE as a poor solvent. The above-mentioned Example 13 corresponds to Embodiment A.

The above-mentioned Example 14 is an example in which cyclic peptide I (C—S type) is produced in Embodiment 2 of the present application by using methanol as a good solvent and IPE as a poor solvent. The above-mentioned Example 14 corresponds to Embodiment B.

The above-mentioned Example 15 is an example in which cyclic peptide G (lactam type) is produced in Embodiment 1 of the present application by using methanol as a good solvent and IPE as a poor solvent.

The above-mentioned Example 16 is an example in which cyclic peptide J (C—S type) is produced in Embodiment 1 of the present application by using chloroform as a good solvent and acetonitrile as a poor solvent. The above-mentioned Example 16 corresponds to Embodiment A.

The above-mentioned Example 17 is an example in which cyclic peptide K (C—S type) is produced in Embodiment 1 of the present application by using chloroform as a good solvent and acetonitrile as a poor solvent. The above-mentioned Example 17 corresponds to Embodiment B.

The above-mentioned Example 18 is an example in which cyclic peptide J (C—S type) is produced in Embodiment 1 of the present application by using methanol as a good solvent and IPE as a poor solvent. The above-mentioned Example 18 corresponds to Embodiment A.

The above-mentioned Example 19 is an example in which cyclic peptide K (C—S type) is produced in Embodiment 1 of the present application by using methanol as a good solvent and IPE as a poor solvent. The above-mentioned Example 19 corresponds to Embodiment B.

### INDUSTRIAL APPLICABILITY

The production method of cyclic peptide of the present invention can efficiently remove multimeric impurity by-produced during a cyclization reaction, improve the purity of the obtained cyclic peptide, and reduce a burden on the purification step.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A method for producing a cyclic peptide, comprising step (1) and step (2):
(1) cyclizing a linear peptide; and
(2) obtaining a cyclic peptide by adding a poor solvent to a mixture of the cyclic peptide and multimeric impurity, which mixture is obtained in the above-mentioned step, and filtering off the multimeric impurity as an insoluble material.

2. The production method according to claim 1, comprising further adding a good solvent before, simultaneously with, or after addition of the above-mentioned poor solvent.

3. The production method according to claim 1, wherein the cyclic peptide has a cyclic structure of either a) S—S type, b) lactam type, c) C—S type, d) C—C type or e) lactone type.

4. The production method according to claim 1, wherein, in the linear peptide, the C-terminal is protected by a protecting group, and cyclization occurs between i) side chains of constituent amino acids or ii) the N-terminal and any of the side chains of the constituent amino acids.

5. The production method according to claim 4, wherein the protecting group of the C-terminal and/or protecting groups of the side chains of the constituent amino acids of the linear peptide are liquid phase protecting groups or pseudo solid phase protecting groups.

6. The production method according to claim 4, wherein, in the above-mentioned step (1), the protecting group of the C-terminal of the linear peptide is a solid phase carrier, and the method further comprises a step of deprotecting the solid phase carrier alone before the above-mentioned step (2).

7. The production method according to claim 4, wherein the poor solvent is a solvent capable of precipitating/depositing multimeric impurities by-produced when obtaining a cyclic peptide by cyclizing the above-mentioned linear peptide.

8. The production method according to claim 7, wherein the good solvent is a solvent capable of dissolving the above-mentioned cyclic peptide of interest.

9. The production method according to claim 1, wherein, in the linear peptide, the C-terminal is not protected, parts other than the part to be cyclized are protected, and cyclization occurs between i) side chains of constituent amino acids, ii) the N-terminal and a side chain of a constituent amino acid, iii) the C-terminal and a side chain of a constituent amino acid, or iv) the N-terminal and the C-terminal.

10. The production method according to claim 9, wherein a protecting group of the side chain of the constituent amino acid of the linear peptide is a liquid phase protecting group or a pseudo solid phase protecting group.

11. The production method according to claim 9, wherein the poor solvent is a solvent capable of precipitating/depositing multimeric impurities by-produced when obtaining a cyclic peptide by cyclizing the above-mentioned linear peptide.

12. The production method according to claim 11, wherein the good solvent is a solvent capable of dissolving the above-mentioned cyclic peptide of interest.

13. The production method according to claim 1, wherein, in the linear peptide, all of the C-terminal, the N-terminal, and the side chains of the constituent amino acids are not protected, and cyclization occurs between i) side chains of constituent amino acids, ii) the N-terminal and a side chain of a constituent amino acid, iii) the C-terminal and a side chain of a constituent amino acid, or iv) the N-terminal and the C-terminal.

14. The production method according to claim 13, further comprising
isolating the cyclic peptide obtained in step (1), between step (1) and step (2).

15. The production method according to claim 13, wherein the poor solvent is a solvent capable of precipitating/depositing multimeric impurities by-produced when obtaining a cyclic peptide by cyclizing the above-mentioned linear peptide.

16. The production method according to claim 15, wherein the good solvent is a solvent capable of dissolving the above-mentioned cyclic peptide of interest.

17. The production method according to claim 1, further comprising
(3) removing all protecting groups after the above-mentioned step (2).

18. A method for producing a peptide having a cyclic thioether bond, comprising any of the following steps:
(A) cyclizing a linear peptide in which the C-terminal of the linear peptide is or is not protected by a protecting group, the N-terminal is modified with an alkylenecarbonyl group having a leaving group, and a side chain of constituent amino acid cysteine or cysteine derivative is not protected, between the N-terminal and the side chain of the cysteine or cysteine derivative,
(B) cyclizing a linear peptide in which the C-terminal of the linear peptide is or is not protected by a protecting group, the N-terminal is or is not protected, and a side chain of constituent amino acid cysteine or cysteine derivative is modified with an alkylene group having a carboxy group, between the N-terminal and the side chain of the cysteine or cysteine derivative, after deprotection of the N-terminal when it is protected, or
(C) cyclizing a linear peptide in which the C-terminal of the linear peptide is or is not protected by a protecting group, an amino group of a side chain of a constituent amino acid is or is not protected, and a side chain of constituent amino acid cysteine or cysteine derivative is modified with an alkylene group having a carboxy group, between the side chain of the constituent amino acid having the amino group and the side chain of the cysteine or cysteine derivative, after deprotection of the amino group of the side chain of the constituent amino acid when it is protected.

19. The production method according to claim 18, wherein the protecting group of the C-terminal of the linear peptide is a pseudo solid phase protecting group, a liquid phase protecting group, or a solid phase carrier.

20. The production method according to claim 18, wherein the step (B) comprises (B-1) producing a linear peptide in which the C-terminal of the linear peptide is or is not protected by a protecting group, the N-terminal is or is not protected, and a side chain of constituent amino acid cysteine or cysteine derivative is modified with an alkylene group having a carboxy group by modifying, with an alkylene group having a carboxy group, a side chain of the constituent amino acid cysteine or cysteine derivative of the linear peptide in which the C-terminal of the linear peptide is or is not protected by a protecting group, the N-terminal is or is not protected, and the side chain of the cysteine or cysteine derivative is not protected, or
the step (C) comprises (C-1) producing a linear peptide in which the C-terminal of the linear peptide is or is not protected by a protecting group, an amino group of a side chain of a constituent amino acid is or is not protected, and a side chain of constituent amino acid cysteine or cysteine derivative is modified with an alkylene group having a carboxy group by modifying, with an alkylene group having a carboxy group, a side chain of constituent amino acid cysteine or cysteine derivative of a linear peptide in which the C-terminal of the linear peptide is or is not protected by a protecting group, an amino group of a side chain of a constituent amino acid is or is not protected, and the side chain of the cysteine or cysteine derivative is not protected.
